(19) United States
(12) Patent Application Publication
(10) Pub. No.: US 2025/0040939 A1
(43) Pub. Date: Feb. 6, 2025

(54) ORTHOPEDIC INSTRUMENTS

(71) Applicant: Paragon 28, Inc., Englewood, CO (US)

(72) Inventors: Daniel J. LEE, Denver, CO (US); Zachary Ryan WOODS, Centennial, CO (US); Mark Ray DALTON, Austin, TX (US); Francis D. BARMES, Parker, CO (US)

(73) Assignee: Paragon 28, Inc., Englewood, CO (US)

(21) Appl. No.: 18/923,361

(22) Filed: Oct. 22, 2024

Related U.S. Application Data
(63) Continuation of application No. PCT/US2023/066225, filed on Apr. 26, 2023.
(60) Provisional application No. 63/363,600, filed on Apr. 26, 2022.

(51) Int. Cl. A61B 17/15; A61B 17/17

(52) U.S. Cl. CPC A61B 17/15; A61B 17/17

(57) ABSTRACT

An orthopedic instrument system includes a resection guide releasably couplable with a bone of a patient. The resection guide includes a pair of bores disposed on an upper portion of the resection guide and extending substantially through the upper portion of the resection guide, and an elongated opening disposed on the upper portion and beneath the pair of bores. The elongated opening includes at least one scalloped opening therein. The resection guide also includes a pair of arms extending substantially downward from the upper portion of the resection guide, wherein each of the arms includes a cut slot that is open in at least one direction. The system also includes a drill guide configured to be inserted into and removed from each of the at least one scalloped opening for the resection guide.

### CROSS REFERENCE TO RELATED APPLICATIONS

This application is a bypass continuation of PCT Application No. PCT/US2023/066225, filed Apr. 26, 2023, and entitled “Orthopedic Instruments,” which claims priority benefit under 35 U.S.C. § 119 (e) of U.S. Provisional Patent Application No. 63/363,600, filed on Apr. 26, 2022, and entitled “Orthopedic Instruments” the disclosures of both applications are hereby incorporated herein by reference in their entirety.

### TECHNICAL FIELD

The present disclosure relates to instruments and corresponding methods associated with performing orthopedic procedures. The present disclosure relates to podiatric and orthopedic implants and surgery related to arthroplasty and/or other orthopedic procedures including but not limited to arthrodesis, arthroeresis and/or osteotomies of joints in the foot/ankle and/or procedures incorporating surrounding bones/soft tissue.

### BACKGROUND OF THE INVENTION

Many currently available instruments, corresponding implant systems, and methods for addressing bone, soft tissue, and joint trauma (acute and chronic, e.g., defect, gradual deterioration, etc.) do not completely address the needs of patients. Additionally, many currently available instruments, implant systems, and methods for procedures to address chronic and acute joint ailments fail to account for properties of joint anatomy and associated mechanical and kinematic movement patterns/capabilities.

### SUMMARY OF THE INVENTION

The present disclosure is directed toward orthopedic instrument systems. According to one aspect of the present disclosure, an orthopedic instrument system includes a resection guide releasably couplable with a bone of a patient. The resection guide includes a pair of bores disposed on an upper portion of the resection guide and extending substantially through the upper portion of the resection guide, and an elongated opening disposed on the upper portion and beneath the pair of bores. The elongated opening includes at least one scalloped opening therein. The resection guide also includes a pair of arms extending substantially downward from the upper portion of the resection guide, wherein each of the arms includes a cut slot that is open in at least one direction. The system also includes a drill guide configured to be inserted into and removed from each of the at least one scalloped opening for the resection guide.

One aspect of the present disclosure is directed to an orthopedic instrument system. The system includes a resection guide releasably couplable with a bone of a patient. The resection guide includes a pair of openings disposed in the resection guide and extending from a top surface through to a bottom surface thereof, an elongated opening disposed in the body and beneath the pair of bores and including at least one scalloped opening therein, and a pair of legs extending substantially downward from the body of the resection guide. Each of the legs includes a slot which is open in at least one direction. The system also includes a drill guide configured to be inserted into an removed from each of the at least one scalloped openings.

According to one aspect of the present disclosure, the pair of legs are positioned at an oblique angle relative to one another.

According to one aspect of the present disclosure, each of the openings includes a central axis, wherein the central axes of the openings are substantially parallel.

According to one aspect of the present disclosure, the system also includes a second pair of openings disposed within the body of the resection guide and extending from the top surface through to the bottom surface thereof.

According to one aspect of the present disclosure, each of the second pair of openings includes a central axis, wherein the central axes of the second pair of openings are either converging or diverging.

According to one aspect of the present disclosure, the elongated opening includes a substantially arced geometry.

According to one aspect of the present disclosure, the resection guide includes a pair of projections extending substantially upward from the body of the resection guide.

According to one aspect of the present disclosure, wherein each of the openings are disposed within each of the projections.

According to one aspect of the present disclosure, the apex of the elongated opening is positioned within the body and laterally between each of the pair of projections.

A second aspect of the present disclosure is directed to a resection guide releasably couplable with a bone of a patient. The resection guide includes a pair of projections extending upward from a body of the resection guide, with each of the projections including an opening disposed therein and extending from a top surface through to the bottom surface of the resection guide. The resection guide also includes an elongated opening disposed in the body and beneath the pair of openings, with the opening including at least one scalloped opening therein. The guide also includes a pair of legs extending substantially downward from the body of the resection guide, with each of the legs including a slot that is open in at least one direction.

According to the second aspect of the present disclosure, the guide includes a second pair of openings disposed within the body of the resection guide.

According to the second aspect of the present disclosure, each opening of the second pair of openings includes a central axis, wherein said central axes are one of converging or diverging.

According to the second aspect of the present disclosure, each of the openings includes a central axis, wherein said central axes are substantially parallel to one another.

According to the second aspect of the present disclosure, the at least one scalloped opening defines at least a portion of a substantially cylindrical geometry extending from the top surface of the resection guide at least partially through to the bottom surface of the resection guide.

According to the second aspect of the present disclosure, the pair of legs extend from the body at a substantially oblique angle.

According to the second aspect of the present disclosure, the elongated opening has a substantially curved geometry.

According to the second aspect of the present disclosure, an apex of the elongated opening is positioned laterally between the pair of projections.

According to the second aspect of the present disclosure, the guide includes a second elongated opening positioned below the elongated opening.

According to the second aspect of the present disclosure, the second elongated opening is defined laterally by each of the pair of legs.

According to the second aspect of the present disclosure, the second elongated opening is configured to receive at least a portion of a cutting tool therein.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a front perspective view of an orthopedic instrument, in accordance with the present disclosure;

FIG. 2 is a front view of the orthopedic instrument of FIG. 1, in accordance with the present disclosure;

FIG. 3 is a rear perspective view of the orthopedic instrument of FIG. 1, in accordance with the present disclosure;

FIG. 4 is a rear view of the orthopedic instrument of FIG. 1, in accordance with the present disclosure;

FIG. 5 is a front perspective view of an orthopedic instrument, in accordance with the present disclosure;

FIG. 6 is a front view of the orthopedic instrument of FIG. 5, in accordance with the present disclosure;

FIG. 7 is a rear perspective view of the orthopedic instrument of FIG. 5, in accordance with the present disclosure;

FIG. 8 is a rear view of the orthopedic instrument of FIG. 5, in accordance with the present disclosure;

FIG. 9 is a front perspective view of an orthopedic instrument, in accordance with the present disclosure;

FIG. 10 is a front view of the orthopedic instrument of FIG. 9, in accordance with the present disclosure;

FIG. 11 is a rear perspective view of the orthopedic instrument of FIG. 9, in accordance with the present disclosure;

FIG. 12 is a rear view of the orthopedic instrument of FIG. 9, in accordance with the present disclosure;

FIG. 13 is a front perspective view of an orthopedic instrument system, in accordance with the present disclosure;

FIG. 14 is a front perspective view of an instrument of the orthopedic instrument system of FIG. 13, in accordance with the present disclosure;

FIG. 15 is a front view of the instrument of FIG. 14 of the orthopedic instrument system of FIG. 13, in accordance with the present disclosure;

FIG. 16 is a rear perspective view of the instrument of FIG. 14 of the orthopedic instrument system of FIG. 13, in accordance with the present disclosure;

FIG. 17 is a rear view of the instrument of FIG. 14 of the orthopedic instrument system of FIG. 13, in accordance with the present disclosure;

FIG. 18 is a front perspective view of an orthopedic instrument of the orthopedic instrument system of FIG. 13, in accordance with the present disclosure;

FIG. 19 is a front view of the orthopedic instrument of FIG. 18 of the orthopedic instrument system of FIG. 13, in accordance with the present disclosure;

FIG. 20 is a rear view of the orthopedic instrument of FIG. 18 of the orthopedic instrument system of FIG. 13, in accordance with the present disclosure;

FIG. 21 is a front perspective view of an orthopedic instrument, in accordance with the present disclosure;

FIG. 22 is a front view of the orthopedic instrument of FIG. 21, in accordance with the present disclosure;

FIG. 23 is a rear perspective view of the orthopedic instrument of FIG. 21, in accordance with the present disclosure;

FIG. 24 is a rear view of the orthopedic instrument of FIG. 21, in accordance with the present disclosure;

FIG. 25 is a front view of a component of an orthopedic instrument system, in accordance with the present disclosure;

FIG. 26 is a front view of the component of the orthopedic instrument system of FIGS. 25, in accordance with the present disclosure;

FIG. 27 is a front view of instruments of the orthopedic instrument system of FIGS. 25-26, in accordance with the present disclosure;

FIG. 28 is a front perspective view of the instruments of FIG. 27 of the orthopedic instrument system of FIGS. 25-26, in accordance with the present disclosure;

FIG. 29 is an alternate front perspective view of the instruments of FIG. 27 of the orthopedic instrument system of FIGS. 25-26, in accordance with the present disclosure;

FIG. 30 is a front view of the components of the instrument system of FIGS. 25-26 in a first position, in accordance with the present disclosure;

FIG. 31 is a front view of the components of the orthopedic instrument system of FIGS. 25-26 in a second position, in accordance with the present disclosure;

FIG. 32 is a front perspective view of an orthopedic instrument system, in accordance with the present disclosure;

FIG. 33 is a front perspective view of an instrument of the orthopedic instrument system of FIG. 32, in accordance with the present disclosure;

FIG. 34 is a front view of the instrument of FIG. 33 of the orthopedic instrument system of FIG. 32, in accordance with the present disclosure;

FIG. 35 is a rear perspective view of the instrument of FIG. 33 of the orthopedic instrument system of FIG. 32, in accordance with the present disclosure;

FIG. 36 is a rear view of the instrument of FIG. 33 of the orthopedic instrument system of FIG. 32, in accordance with the present disclosure;

FIG. 37 is a front perspective view of the instruments of the orthopedic instrument of system of FIG. 32, in accordance with the present disclosure;

FIG. 38 is a front view of instruments of the orthopedic instrument system of FIG. 32, in accordance with the present disclosure;

FIG. 39 is a front view of an orthopedic instrument of the orthopedic instrument system of FIG. 32, in accordance with the present disclosure;

FIG. 40 is a front perspective view of the orthopedic instrument of FIG. 39 of the orthopedic instrument system of FIG. 32, in accordance with the present disclosure;

FIG. 41 is a rear view of the orthopedic instrument of FIG. 39 of the orthopedic instrument system of FIG. 32, in accordance with the present disclosure;

FIG. 42 is a front view of components of the orthopedic instrument system of FIG. 32, in accordance with the present disclosure;

FIG. 43 is a front perspective view of an orthopedic instrument, in accordance with the present disclosure;

FIG. 44 is a front view of the orthopedic instrument of FIG. 43, in accordance with the present disclosure;

FIG. 45 is a rear perspective view of the orthopedic instrument of FIG. 43, in accordance with the present disclosure;

FIG. 46 is a rear view of the orthopedic instrument of FIG. 43, in accordance with the present disclosure;

FIG. 47 is a front view of an orthopedic instrument, in accordance with the present disclosure;

FIG. 48 is a front perspective view of the orthopedic instrument of FIG. 47, in accordance with the present disclosure;

FIG. 49 is a rear view of the orthopedic instrument of FIG. 47, in accordance with the present disclosure;

FIG. 50 is a front perspective view of an orthopedic instrument system, in accordance with the present disclosure;

FIG. 51 is a front view of the instruments of the orthopedic instrument system of FIG. 50, in accordance with the present disclosure;

FIG. 52 is a front perspective view of an instrument of the orthopedic instrument system of FIG. 50, in accordance with the present disclosure;

FIG. 53 is a front view of the instrument of FIG. 52 of the orthopedic instrument system of FIG. 50, in accordance with the present disclosure;

FIG. 54 is a rear perspective view of the instrument of FIG. 52 of the orthopedic instrument system of FIG. 50, in accordance with the present disclosure;

FIG. 55 is a rear view of the instrument of FIG. 52 of the orthopedic instrument system of FIG. 50, in accordance with the present disclosure;

FIG. 56 is a front view of an instrument of the orthopedic instrument system of FIG. 50, in accordance with the present disclosure;

FIG. 57 is a front perspective view of the orthopedic instrument of FIG. 56 of the orthopedic instrument system of FIG. 50, in accordance with the present disclosure;

FIG. 58 is a rear view of the orthopedic instrument of FIG. 56 of the orthopedic instrument system of FIG. 50, in accordance with the present disclosure;

FIG. 59 is a front perspective view of the components of the orthopedic instrument system of FIG. 50, in accordance with the present disclosure;

FIG. 60 is a front perspective view of an orthopedic instrument, in accordance with the present disclosure;

FIG. 61 is a front view of the orthopedic instrument of FIG. 60, in accordance with the present disclosure;

FIG. 62 is a rear perspective view of the orthopedic instrument of FIG. 60, in accordance with the present disclosure;

FIG. 63 is a rear view of the orthopedic instrument of FIG. 60, in accordance with the present disclosure;

FIG. 64 is a front view of an orthopedic instrument, in accordance with the present disclosure;

FIG. 65 is a rear perspective view of the orthopedic instrument of FIG. 64, in accordance with the present disclosure;

FIG. 66 is a rear view of the orthopedic instrument of FIG. 64, in accordance with the present disclosure;

FIG. 67 is a perspective view of an orthopedic instrument, in accordance with the present disclosure;

FIG. 68 is a front perspective view of an orthopedic instrument, in accordance with the present disclosure;

FIG. 69 is a front view of the orthopedic instrument of FIG. 68, in accordance with the present disclosure;

FIG. 70 is a rear perspective view of the orthopedic instrument of FIG. 68, in accordance with the present disclosure; and

FIG. 71 is a rear view of the orthopedic instrument of FIG. 68, in accordance with the present disclosure.

### DETAILED DESCRIPTION OF THE INVENTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

This application relates to U.S. Continuation application Ser. No. 17/659,411 filed on Apr. 15, 2022, entitled Joint Replacement Alignment Guides, Systems and Methods of Use and Assembly (Attorney Docket No. 3645.155B), which is a continuation of U.S. application Ser. No. 17/304,046 filed Jun. 14, 2021 (now U.S. Pat. No. 11,304,823) and entitled Joint Replacement Alignment Guides, Systems and Methods of Use and Assembly (Attorney Docket No. 3645.155A), which is a continuation of PCT/US2019/066408 filed on Dec. 13, 2019 and entitled Joint Replacement Alignment Guides, Systems and Methods of Use and Assembly, which claims priority benefit under 35 U.S.C. § 119 (e) of U.S. Provisional Application No. 62/899,703 filed Sep. 12, 2019 and entitled Joint Replacement Alignment Guides, Systems and Methods of Use and Assembly (Attorney Docket No. 3645.155P1), U.S. Provisional Application No. 62/899,655 filed Sep. 12, 2019 and entitled Alignment Instruments and Methods for Use in Total Ankle Replacement (Attorney Docket No. 3645.156P1), U.S. Provisional Application No. 62/899,740 filed Sep. 12, 2019 and entitled Joint Replacement Alignment Guides, Systems and Methods of Use and Assembly (Attorney Docket No. 3645.157P1), and U.S. Provisional Application No. 62/779,436 filed Dec. 13, 2018 and entitled Joint Replacement Systems and Methods of Use and Assembly (Attorney Docket No. 3645.138P), which are all incorporated herein by reference in their entireties.

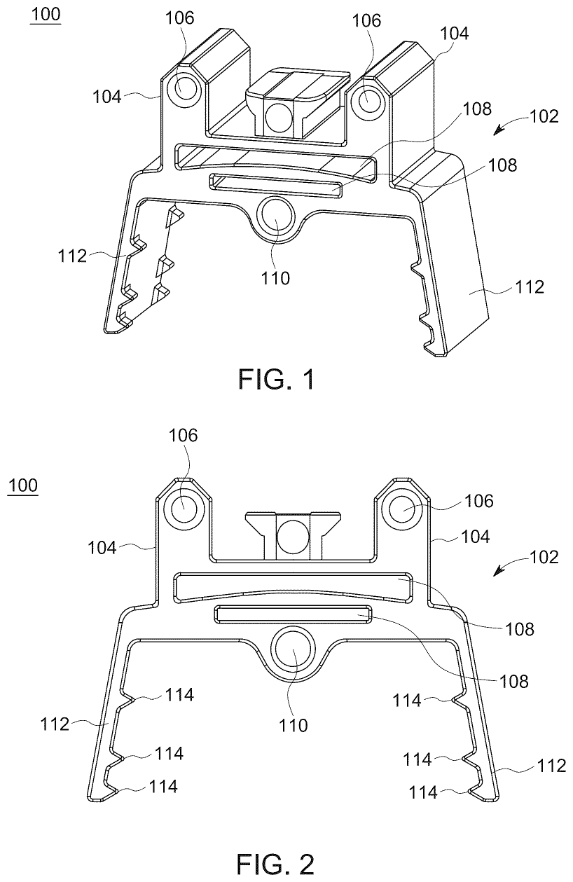

Referring now to FIGS. 1-71, orthopedic instrument systems and components thereof are shown, according to various aspects of the present disclosure. In some embodiments, one or more instruments of the components showed and described herein may be releasably coupled with one or more bones of a patient, for example a tibia and/or talus of a patient. One or more pins, stabilization wires, or other components may be implemented in this releasable coupling. For example, the one or more pins or stabilization wire may be inserted into and/or at least partially through one or more bores extending through a portion of the attached instruments. In some aspects, the instruments shown and described herein may include bores with parallel longitudinal axes and/or include bores with converging and/or diverging longitudinal axes. Further, various instruments shown in FIGS. 1-71 may also include other coupling features, for example releasable coupling components disposed on an upper portion of resection and/or alignment guides. Further to the previous example, components may include a dovetail feature (e.g., as shown, a male dovetail feature but may also include other coupling mechanisms) configured to interface with a corresponding complimentary feature of an alignment system or other surgical instrument/component.

Some of the instruments shown in FIGS. 1-71 may include portions thereof configured to receive one or multiple drills one or more times over the course of performing a method that corresponds to a portion of a surgical procedure. For example, the instruments shown and described herein may include an elongated opening extending along an upper portion of an instrument and extending from a front portion of the instrument through to a back portion of the instrument so as to provide fluid communication between the front and back of the instrument. In some aspects, the elongated opening (which may be substantially linear and/or include a curvature) may include one or more scalloped openings (e.g., partially defined geometries within the elongated opening) which, for example, may include a partially circular geometry (e.g., have a radius along a portion but not an entirety of a substantially circular geometry). The elongated opening and/or the scalloped openings may be configured to receive one or more instruments therein, for example guides to guide a drill used to drill holes in a bone of a patient. In some aspects, an insert (or multiple inserts sequentially) may be placed within the entirety of the elongated opening with multiple geometries to guide a drill. Further, in some aspects a single instrument (e.g., a single-barrel drill guide) such as shown and described herein may be placed within one or more of the scallops (e.g., scalloped openings) with a drill bit then inserted within the barrel, a hole drilled in a bone of a patient, the drill bit and guide removed, and the process repeated with another scalloped opening. In some aspects, this process may be repeated multiple times across the length of the elongated opening through various scalloped openings.

Various instruments shown and described with reference to FIGS. 1-71 may also include one or more legs extending in a substantially downward direction from an upper portion of the instrument (e.g., extending from a point below and/or beyond the lateral-most edges of the elongated opening as such components have been described previously). In some aspects, the legs may include one or more cut slots configured to receive at least a portion of a cutting instrument common to orthopedic procedures (e.g., various types of saws, etc.). As shown in various aspects of the FIGS. 1-71, the cut slots disposed on the legs of said instruments may be open sided in at least one dimension (e.g., a substantially rectangular cut slot open on at least one of the four sides, a hexagonal cut slot open on at least one of the six sides, etc.). In some aspects, the cut slots may extend into various portions of the instrument to allow for pivoting of a saw to cut a desired portion of a bone of a patient (e.g., a tibia). For example, a cut slot disposed on a leg of an instrument may extend at a partial depth of the instrument into an upper portion of said instrument adjacent the aforementioned elongated opening.

The various aspects of instruments shown and described herein may include various reference points or markers configured to aid a physician in placing said instrument and/or other complimentary instruments relative to anatomy of a patient over the course of a procedure. For example, an instrument may include a crossbar configured to indicate varus/valgus alignment of a resection guide or other components. Similarly, an instrument may include one or more pointers or markers (e.g., angled protrusions, etc.) configured to be aligned with components of other instruments of an instrument system and/or anatomical reference points of a patient. In some aspects, an instrument may include multiple reference points or markers, with said reference points or markers of various shapes, sizes, etc. and configured to aid alignment of various components (e.g., instruments, anatomy, etc.) in various directions (e.g., varus-valgus, superior-inferior, medial-lateral, anterior-posterior, distal-proximal, plantar-dorsal, etc.).

FIGS. 1-71 may also show instruments configured to make cuts adjacent to those described with reference to the cut slots disposed on legs of instruments as described previously herein. For example, an instrument may include an upper portion configured to couple with a bone of a patient in a manner the same and/or similar to that described previously. Further, an instrument may be configured to couple with a first bone of a patient and extend to a position adjacent a second bone of a patient, where said instrument is configured to facilitate (e.g., guide, etc.) a resection cut to said second bone of the patient. In some aspects, such an instrument may include one or more reference points or markers disposed between an upper portion (e.g., a couplable portion) and a terminal end of an extension extending from said upper portion in a substantially downward direction. Said terminal end may include an elongated component with one or more cut slots disposed at least partially thereon. In some aspects, said one or more cut slots may be open in at least one dimension (e.g., where “open” can be defined just as defined previously in a similar context). Further, in some aspects such an instrument may be reversible. For example, said instrument may be configured to be coupled with a bone of a patient, a first cut made, said guide decoupled and recoupled in a reverse configuration (e.g., where the said open dimension of the slot faces an opposite direction), with a second cut made adjacent to and/or overlapping the first cut.

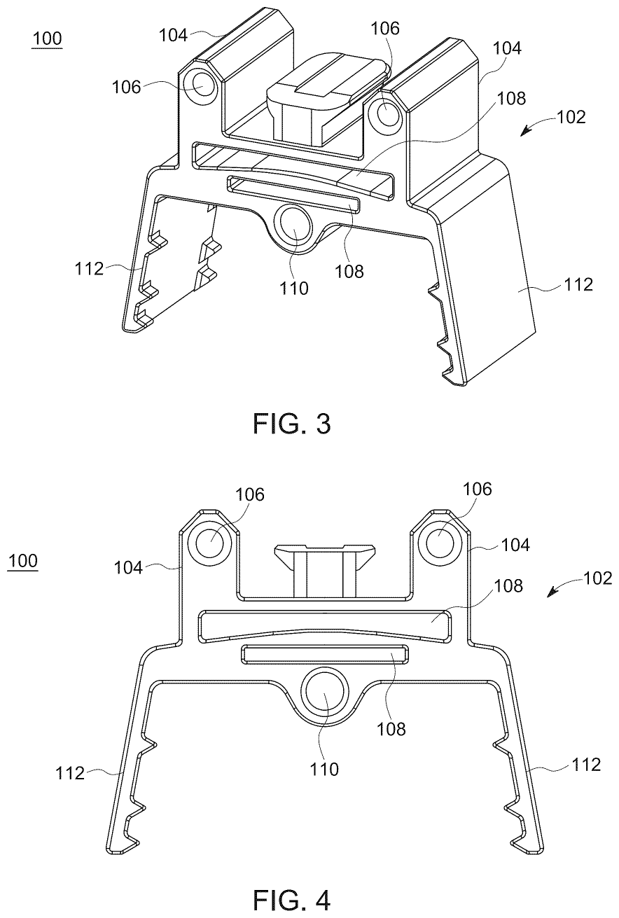

Referring now to FIGS. 1-4, a guide 100 is shown, according to an exemplary embodiment. The guide 100 is shown to include a body 102, with a pair of projections 104 extending upward from the body 102. As shown, the projections 104 extend upward from the body 102 substantially parallel to one another and, as shown, the projections 104 include substantially the same geometry (although in some aspects the projections 104 may include alternate geometries from that shown and/or from one another). Each of the projections 104 are shown to include an opening 106 (e.g., a bore, etc.) centrally positioned relative to the projection 104 and extending from a top surface of the guide 100 through to the bottom surface. In some aspects, one or more of the openings 106 may be biased toward a terminal end of the projections 104. As shown, each of the openings 106 include substantially the same size and positioning relative to the projections 104 and the body 102. However, in some aspects the openings 106 may include alternate sizing and/or positioning. As shown, each of the openings 106 are positioned within the projections 104, however in some aspects one or more openings 106 may be positioned within the body 102 adjacent to the base of the projections 104. In some aspects, one or more pins may be placed within and/or through the openings 106 and into a bone (e.g., a tibia) so as to couple the guide 100 with the bone.

The body 102 is further shown to include openings 108 as well as an opening 110 disposed in a central portion thereof. As shown, the openings 108 are configured as substantially elongated, rectangular geometry/slots and are positioned above the opening 110. The opening 110 is shown to include a substantially cylindrical geometry and is positioned below the openings 108. As shown, the openings 108, 110 extend from a top surface through to a bottom surface of the guide 100. In some aspects, the openings 108 may be configured to receive at least a portion of an elongated component therein, for example an angel wing or other alignment aid. Similarly, the opening 110 may also be configured to receive a component therein and/or therethrough, for example a pin, drill guide and drill bit, or other common surgical component with a geometry complimentary to that of the opening 110.

The guide 100 is further shown to include a pair of legs 112 extending downward (e.g., in a direction opposite that of the projections) from opposing lateral sides of the body 102. As shown, legs 112 extend at a substantially oblique angle relative to one another. Further, each of the legs 112 (at a point along a length thereof, for example the terminal end) defines the greatest lateral dimension of the guide 100 on each side thereof. Each of the legs 112 are shown to include at least one projection 114 on an inner surface thereof. As shown, the projections 114 are substantially triangular in shape and points toward an opening positioned between the legs 112 and defined laterally by the legs 112.

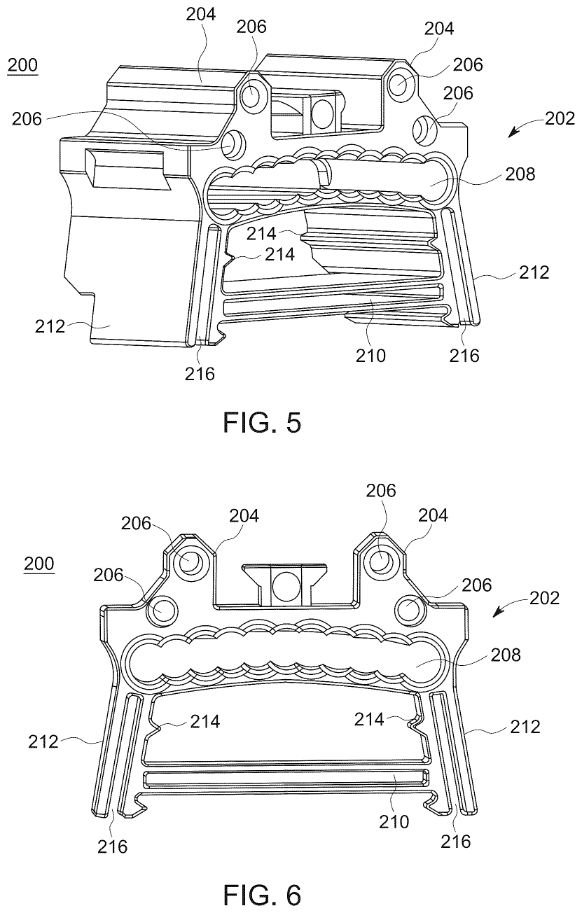

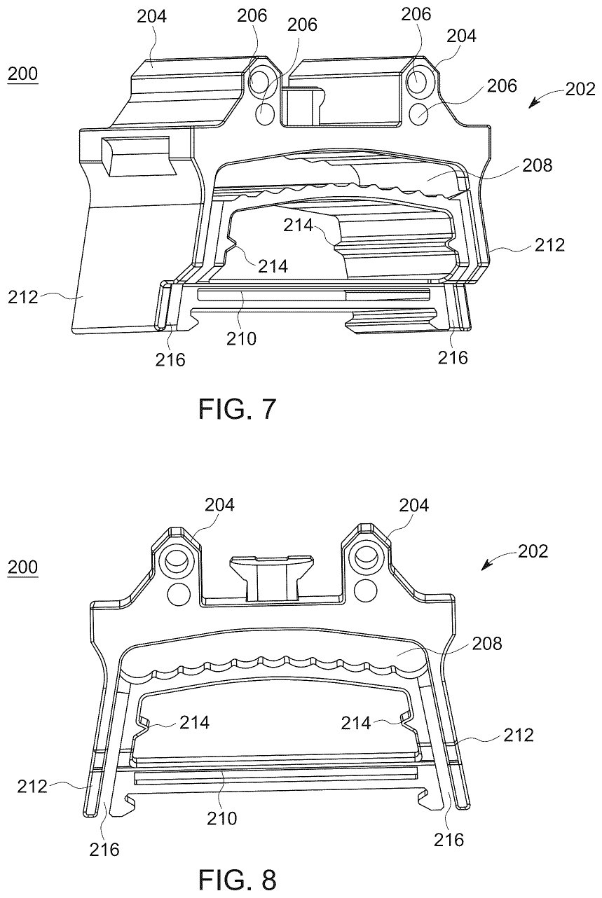

Referring now to FIGS. 5-8, a guide 200 is shown, according to an exemplary embodiment. The guide 200 is shown to include a body 202, with a pair of projections 204 extending upward from the body 202. As shown, the projections 204 extend upward from the body 202 substantially parallel to one another and, as shown, the projections 204 include substantially the same geometry (although in some aspects the projections 204 may include alternate geometries from that shown and/or from one another). Each of the projections 204 are shown to include at least one opening 206 (e.g., a bore, etc.), where at least one of the openings 206 is centrally positioned relative to the projection 204 and extending from a top surface of the guide through to the bottom surface. In some aspects, one or more of the openings 206 may be biased toward a terminal end of the projections 204. In some aspects, a second pair of openings 206 may be disposed within the body 202 of the guide 200. As shown, the openings 206 are disposed within the projections 204 may have parallel central axes, whereas the second pair of openings 206 (disposed in the body 202) may have converging (or diverging) central axes. As shown, each of the openings 206 include substantially the same size and positioning relative to the projections 204 and the body 202. However, in some aspects the openings 206 may include alternate sizing and/or positioning. As shown, at least two of the openings 206 are positioned within the projections 204, however in some aspects one or more openings 206 may be positioned within the body 202 adjacent to the base of the projections 204. In some aspects, one or more pins may be placed within and/or through the openings 206 and into a bone (e.g., a tibia) so as to couple the guide 200 with the bone. In some aspects, the guide 200 may include a coupling element (e.g., a male or female dovetail component, etc.) extending from a portion of the guide 200 and configured to facilitate releasably coupling of the guide 200 with other instrumentation and components a system, for example an ankle arthroplasty system.

The body 202 is further shown to include an opening 208 disposed in a central portion thereof. The opening 208 is shown to have a substantially arced geometry (e.g., curved, radiused, etc.) where an apex of the arced geometry is positioned laterally between the projections 204. Further, the opening 208 is shown to include a plurality of scallops disposed within, where the scallops extend from at least one of upper, lower, and lateral sides of the opening 208, with each of the scallops defining at least a portion (but not necessarily an entirety) of a cylindrical geometry (or other similar geometry) extending from the top surface of the guide 200 at least partially through to the bottom surface (e.g., through the depth of the opening 208). Accordingly, a drill guide (including but not limited to those shown subsequently herein) having a complimentary size and geometry to the scallops (e.g., a cylindrical drill guide) may be inserted within each of the scallops so as to guide a drill therein and therethrough such that drilling into the bone with which the guide 200 is coupled may be accomplished. The opening 208 includes a substantially elongated, rectangular geometry and is positioned above a central window defined on all sides by components of the guide 200, with the central window configured to provide visibility of the anatomy (e.g., a joint) beneath the guide 200 when the guide 200 is coupled with the bone.

The guide 200 is further shown to include a pair of legs 212 extending downward (e.g., in a direction opposite that of the projections) from opposing lateral sides of the body 202. As shown, legs 212 extend at a substantially oblique angle relative to one another. Further, each of the legs 212 (at a point along a length thereof, for example the terminal end) defines the greatest lateral dimension of the guide 200 on each side thereof. Each of the legs 212 are shown to include at least one projection 214 on an inner surface thereof. As shown, at least one of the projections 214 is substantially triangular in shape and points toward the central opening positioned between the legs 212 and defined laterally by the legs 212. Each of the legs is shown to include a slot 216 disposed therein, wherein the slot 216 is defined laterally by each of the legs 212 and vertically in an upward direction by the body 202, but is undefined vertically in a lower direction. Accordingly, each of the slots 216 may be configured to receive a cutting instrument therein (e.g., a reciprocating saw, sagittal saw, etc.) so as to facilitate cutting/resection of the bone with which the guide 200 is coupled. As shown, the slots 216 are positioned substantially parallel to a central axis of each of the legs 212, however in some aspects the slots 216 may include alternate geometries.

The guide 200 is further shown to include an opening 210 defined laterally by the pair of legs 212 and vertically by a pair of cross members, with each of the cross members being integral with the pair of legs 212. The opening 210 may be configured to receive a surgical instrument therein, for example a cutting instrument (e.g., reciprocating saw, sagittal saw, etc.) so as to guide a cut in a second bone, where the second bone is adjacent the bone with which the projections 204 may be coupled. As shown, the opening 210 is a substantially horizontal opening and positioned in a plane parallel to a plane that is tangent to the arced geometry of the opening 208, however in some aspects the opening 210 may have alternate geometries.

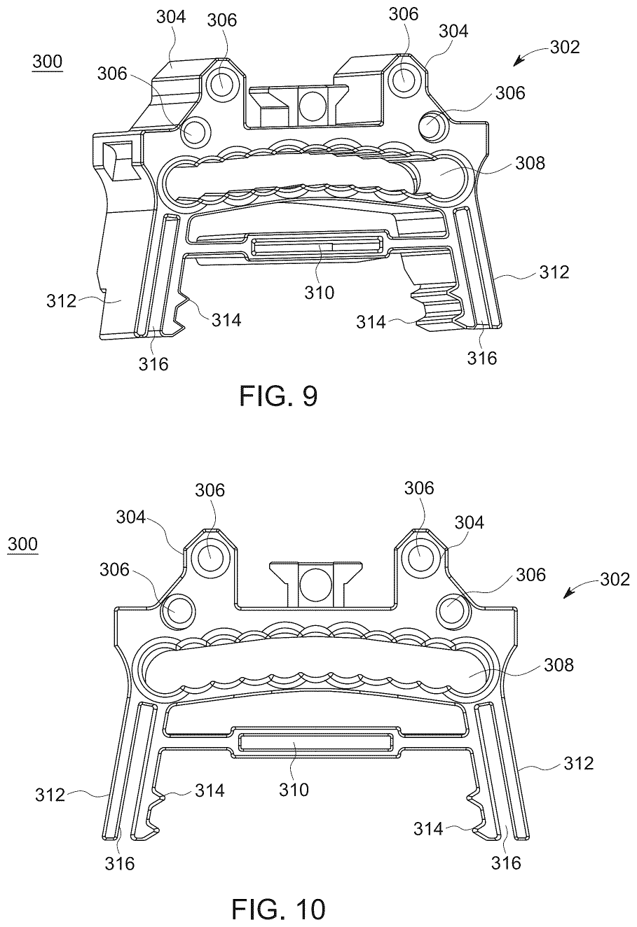

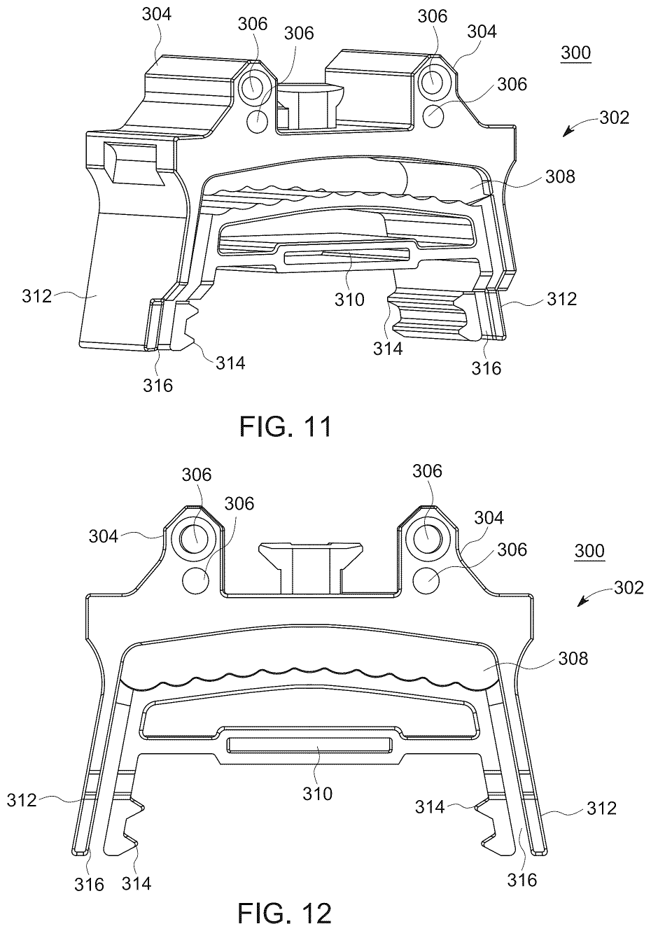

Referring now to FIGS. 9-12, a guide 300 is shown, according to an exemplary embodiment. The guide 300 is shown to include a body 302, with a pair of projections 304 extending upward from the body 302. As shown, the projections 304 extend upward from the body 302 substantially parallel to one another and, as shown, the projections 304 include substantially the same geometry (although in some aspects the projections 304 may include alternate geometries from that shown and/or from one another). Each of the projections 304 are shown to include at least one opening 306 (e.g., a bore, etc.), where at least one of the openings 306 is centrally positioned relative to the projection 304 and extending from a top surface of the guide through to the bottom surface. In some aspects, one or more of the openings 306 may be biased toward a terminal end of the projections 304. In some aspects, a second pair of openings 306 may be disposed within the body 302 of the guide 300 and/or partially within the body 302 of the guide 200 and partially within the projection 304. As shown, the openings 306 disposed within the projections 304 may have parallel central axes, whereas the second pair of openings 306 (disposed in the body 302) may have converging (or diverging) central axes. As shown, each of the openings 306 include substantially the same size and positioning relative to the projections 304 and the body 302. However, in some aspects the openings 306 may include alternate sizing and/or positioning. As shown, at least two of the openings 306 are positioned within the projections 304, however in some aspects one or more openings 306 may be positioned within the body 302 adjacent to the base of the projections 304. In some aspects, one or more pins may be placed within and/or through the openings 306 and into a bone (e.g., a tibia) so as to couple the guide 300 with the bone. In some aspects, the guide 300 may include a coupling element (e.g., a male or female dovetail component, etc.) extending from a portion of the guide 300 and configured to facilitate releasably coupling of the guide 300 with other instrumentation and components a system, for example an ankle arthroplasty system.

The body 302 is further shown to include an opening 308 disposed in a central portion thereof. The opening 308 is shown to have a substantially arced geometry (e.g., curved, radiused, etc.) where an apex of the arced geometry is positioned laterally between the projections 304. Further, the opening 308 is shown to include a plurality of scallops disposed within, where the scallops extend from at least one of upper, lower, and lateral sides of the opening 308, with each of the scallops defining at least a portion (but not necessarily an entirety) of a cylindrical geometry (or other similar geometry) extending from the top surface of the guide 300 at least partially through to the bottom surface (e.g., through the depth of the opening 308). Accordingly, a drill guide (including but not limited to those shown subsequently herein) having a complimentary size and geometry to the scallops (e.g., a cylindrical drill guide) may be inserted within each of the scallops so as to guide a drill therein and therethrough such that drilling into the bone with which the guide 300 is coupled may be accomplished. The opening 308 includes a substantially elongated, rectangular geometry and is positioned above a central window defined on all sides by components of the guide 300, with the central window configured to provide visibility of the anatomy (e.g., a joint) beneath the guide 300 when the guide 300 is coupled with the bone.

The guide 300 is further shown to include a pair of legs 312 extending downward (e.g., in a direction opposite that of the projections) from opposing lateral sides of the body 302. As shown, legs 312 extend at a substantially oblique angle relative to one another. Further, each of the legs 312 (at a point along a length thereof, for example the terminal end) defines the greatest lateral dimension of the guide 300 on each side thereof. Each of the legs 312 are shown to include at least one projection 314 on an inner surface thereof. As shown, at least one of the projections 314 is substantially triangular in shape and points toward the central opening positioned between the legs 312 and defined laterally by the legs 312. Each of the legs is shown to include a slot 316 disposed therein, wherein the slot 316 is defined laterally by each of the legs 312 and vertically in an upward direction by the body 302, but is undefined vertically in a lower direction. Accordingly, each of the slots 316 may be configured to receive a cutting instrument therein (e.g., a reciprocating saw, sagittal saw, etc.) so as to facilitate cutting/resection of the bone with which the guide 300 is coupled. As shown, the slots 316 are positioned substantially parallel to a central axis of each of the legs 312, however in some aspects the slots 316 may include alternate geometries.

The guide 300 is further shown to include an opening 310 disposed within a cross member extending laterally between the pair of legs 312. The opening 310 is disposed entirely within the cross member with the cross member being integral with the pair of legs 312. The opening 310 may be configured to receive a surgical instrument therein, for example an alignment instrument such as an angelwing or other similar component. As shown, the opening 310 is a substantially horizontal and positioned in a plane parallel to a plane that is tangent to the arced geometry of the opening 308, however in some aspects the opening 310 may have alternate geometries.

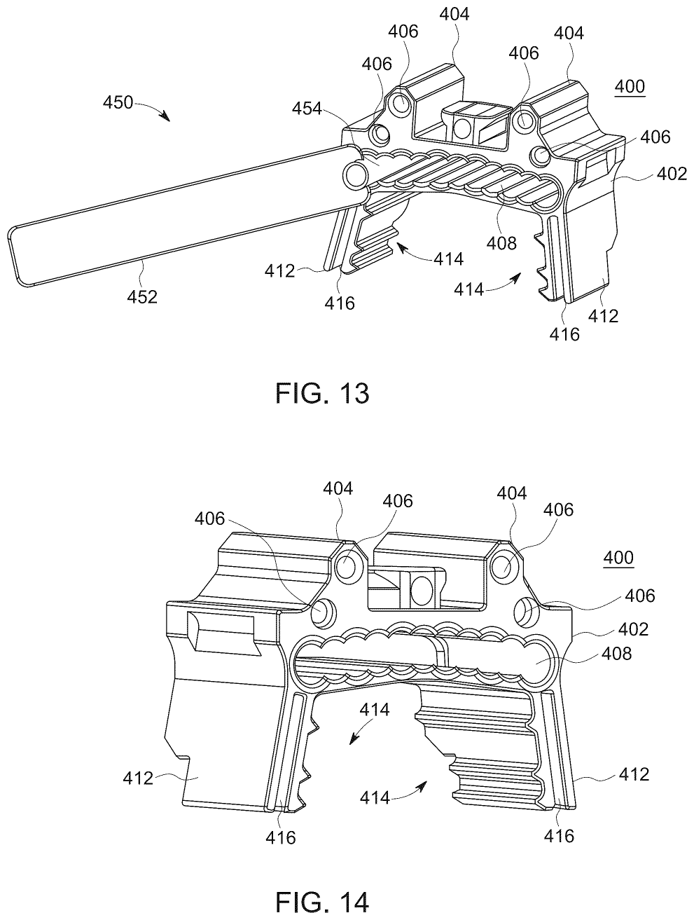

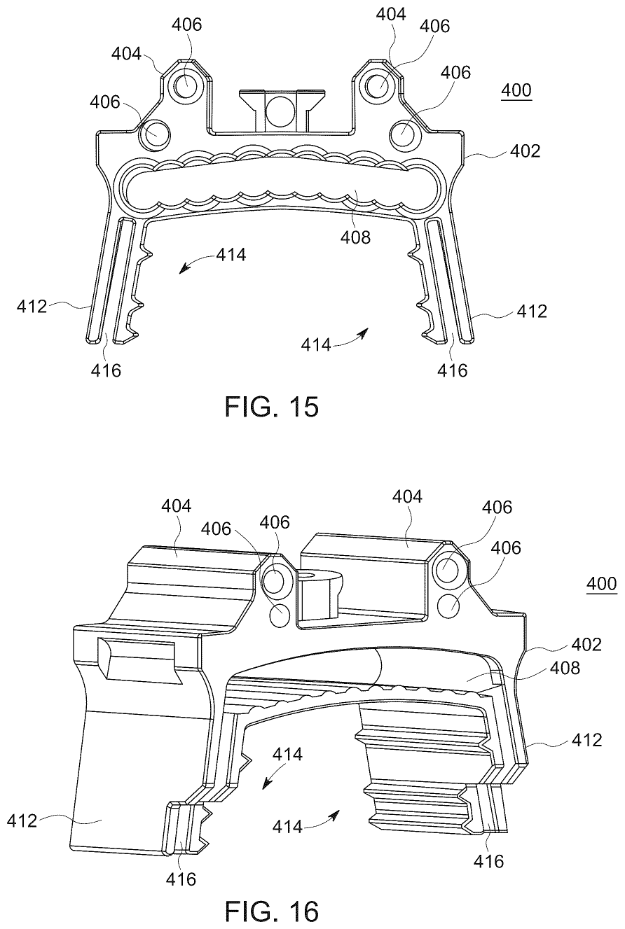

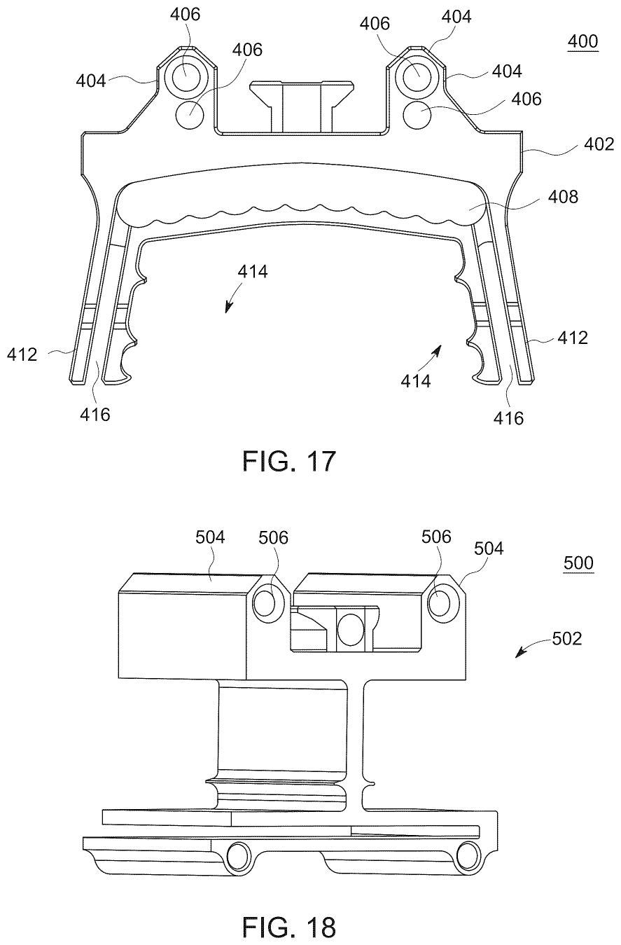

Referring now to FIGS. 13-17, a guide 400 is shown, according to an exemplary embodiment. The guide 400 is shown to include a body 402, with a pair of projections 404 extending upward from the body 402. As shown, the projections 404 extend upward from the body 402 substantially parallel to one another and, as shown, the projections 404 include substantially the same geometry (although in some aspects the projections 404 may include alternate geometries from that shown and/or from one another). Each of the projections 404 are shown to include at least one opening 406 (e.g., a bore, etc.), where at least one of the openings 406 is centrally positioned relative to the projection 404 and extending from a top surface of the guide through to the bottom surface. In some aspects, one or more of the openings 406 may be biased toward a terminal end of the projections 404. In some aspects, a second pair of openings 406 may be disposed within the body 402 of the guide 400 and/or partially within the body 402 of the guide 400 and partially within the projection 404. As shown, the openings 406 disposed within the projections 404 may have parallel central axes, whereas the second pair of openings 406 (disposed in the body 402) may have converging (or diverging) central axes. As shown, each of the openings 406 include substantially the same size and positioning relative to the projections 404 and the body 402. However, in some aspects the openings 406 may include alternate sizing and/or positioning. As shown, at least two of the openings 406 are positioned within the projections 404, however in some aspects one or more openings 406 may be positioned within the body 402 adjacent to the base of the projections 404. In some aspects, one or more pins may be placed within and/or through the openings 406 and into a bone (e.g., a tibia) so as to couple the guide 400 with the bone. In some aspects, the guide 400 may include a coupling element (e.g., a male or female dovetail component, etc.) extending from a portion of the guide 400 and configured to facilitate releasably coupling of the guide 400 with other instrumentation and components a system, for example an ankle arthroplasty system.

The body 402 is further shown to include an opening 408 disposed in a central portion thereof. The opening 408 is shown to have a substantially arced geometry (e.g., curved, radiused, etc.) where an apex of the arced geometry is positioned laterally between the projections 404. Further, the opening 408 is shown to include a plurality of scallops disposed within, where the scallops extend from at least one of upper, lower, and lateral sides of the opening 408, with each of the scallops defining at least a portion (but not necessarily an entirety) of a cylindrical geometry (or other similar geometry) extending from the top surface of the guide 400 at least partially through to the bottom surface (e.g., through the depth of the opening 408). Accordingly, a drill guide (including but not limited to those shown subsequently herein) having a complimentary size and geometry to the scallops (e.g., a cylindrical drill guide) may be inserted within each of the scallops so as to guide a drill therein and therethrough such that drilling into the bone with which the guide 400 is coupled may be accomplished. The opening 408 includes a substantially elongated, rectangular geometry and is positioned above a central window defined on all sides by components of the guide 400, with the central window configured to provide visibility of the anatomy (e.g., a joint) beneath the guide 400 when the guide 400 is coupled with a bone.

The guide 400 is further shown to include a pair of legs 412 extending downward (e.g., in a direction opposite that of the projections) from opposing lateral sides of the body 402. As shown, legs 412 extend at a substantially oblique angle relative to one another. Further, each of the legs 412 (at a point along a length thereof, for example the terminal end) defines the greatest lateral dimension of the guide 400 on each side thereof. Each of the legs 412 are shown to include at least one projection 414 on an inner surface thereof. As shown, at least one of the projections 414 is substantially triangular in shape and points toward the central opening positioned between the legs 412 and defined laterally by the legs 412. Each of the legs is shown to include a slot 416 disposed therein, wherein the slot 416 is defined laterally by each of the legs 412 and vertically in an upward direction by the body 402, but is undefined vertically in a lower direction. Accordingly, each of the slots 416 may be configured to receive a cutting instrument therein (e.g., a reciprocating saw, sagittal saw, etc.) so as to facilitate the cutting/resecting of the bone with which the guide 400 is coupled. As shown, the slots 416 are positioned substantially parallel to a central axis of each of the legs 412, however in some aspects the slots 416 may include alternate geometries.

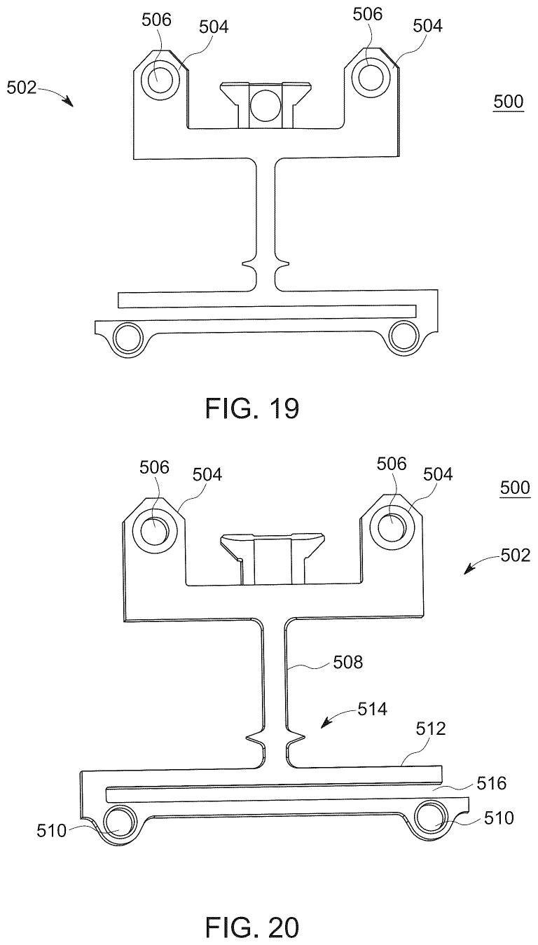

Referring now to FIGS. 18-20, a guide 500 is shown, according to an exemplary embodiment. The guide 500 is shown to include a body 502, with a pair of projections 504 extending upward from the body 502. As shown, the projections 504 extend upward from the body 502 substantially parallel to one another and, as shown, the projections 504 include substantially the same geometry (although in some aspects the projections 504 may include alternate geometries from that shown and/or from one another). Each of the projections 504 are shown to include at least one opening 506 (e.g., a bore, etc.), where at least one of the openings 506 is centrally positioned relative to the projection 504 and extending from a top surface of the guide through to the bottom surface. In some aspects, one or more of the openings 506 may be biased toward a terminal end of the projections 504. In some aspects, a second pair of openings 506 may be disposed within the body 502 of the guide 500 and/or partially within the body 502 of the guide 500 and partially within the projection 504. As shown, the openings 506 disposed within the projections 504 may have parallel central axes, whereas the second pair of openings 506 (disposed in the body 502) may have converging (or diverging) central axes. As shown, each of the openings 506 include substantially the same size and positioning relative to the projections 504 and the body 502. However, in some aspects the openings 506 may include alternate sizing and/or positioning. As shown, at least two of the openings 506 are positioned within the projections 504, however in some aspects one or more openings 506 may be positioned within the body 502 adjacent to the base of the projections 504. In some aspects, one or more pins may be placed within and/or through the openings 506 and into a bone (e.g., a tibia) so as to couple the guide 500 with the bone. In some aspects, the guide 500 may include a coupling element (e.g., a male or female dovetail component, etc.) extending from a portion of the guide 500 and configured to facilitate releasably coupling of the guide 500 with other instrumentation and components of a system, for example an ankle arthroplasty system.

The guide 500 is further shown to include a shaft 508 extending downward from the body 502 to a frame 512 disposed substantially below the body 502. As shown, the body 502, shaft 508, and frame 512 are integral with one another with the shaft 508 extending from both the body 502 and the frame 512 at a substantially orthogonal angle from surfaces of the respective components. The shaft 508 is shown to include at least one projection 514 on an outer surface thereof. As shown, at least one of the projections 514 is substantially triangular in shape and points outward from a surface of the shaft 508 in a substantially orthogonal direction. The frame 512 is shown to include a pair of openings 510 on a lower portion thereof, with the openings configured to facilitate the coupling of the frame 512 with a second bone of a patient (for example, a talus). As shown, the openings 510 are positioned below a slot 516 disposed within the frame 512, where the slot 516 is defined in vertical directions (upward and downward) and on a lateral side by the frame 512. Further, the slot 516 is undefined or open on one side (of a rectangular geometry, as shown) so as to facilitate the insertion and removal of a cutting instrument, for example a reciprocating or sagittal saw. In some aspects the guide 500 may be reversible, such that the guide 500 may be placed over pins received in the openings 506, 510, a cut made within the slot 516, then the guide 500 removed, flipped 180 degrees, and repositioned with pins received in the openings 506, 510 and the open side of the slot 516 facing an opposite direction from the initial position so as to facilitate an extension of the first cut or guidance of a second cut.

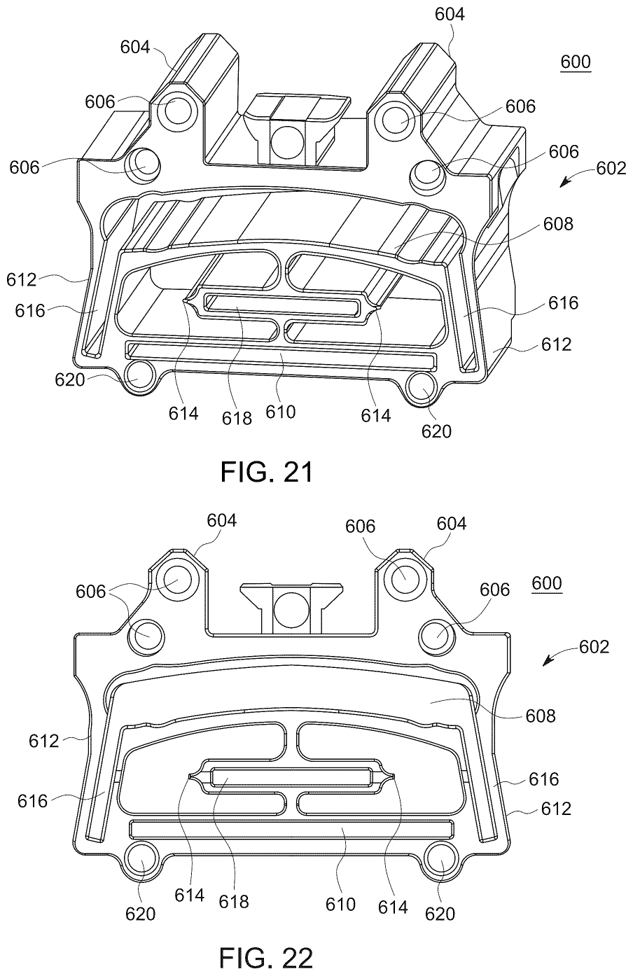

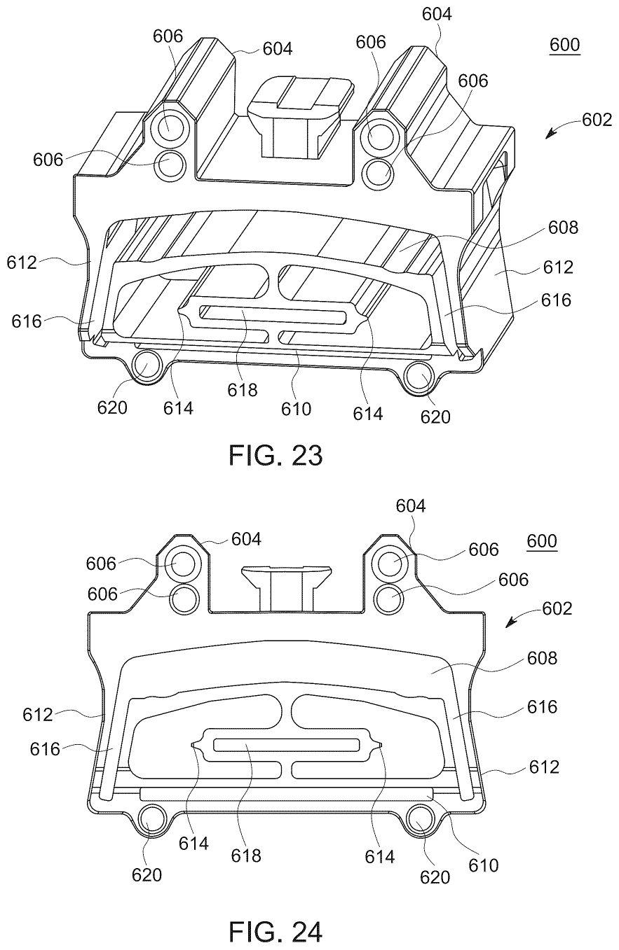

Referring now to FIGS. 21-24, a guide 600 is shown, according to an exemplary embodiment. The guide 600 is shown to include a body 602, with a pair of projections 604 extending upward from the body 602. As shown, the projections 604 extend upward from the body 602 substantially parallel to one another and, as shown, the projections 604 include substantially the same geometry (although in some aspects the projections 604 may include alternate geometries from that shown and/or from one another). Each of the projections 604 are shown to include at least one opening 606 (e.g., a bore, etc.), where at least one of the openings 606 is centrally positioned relative to the projection 604 and extending from a top surface of the guide through to the bottom surface. In some aspects, one or more of the openings 606 may be biased toward a terminal end of the projections 604. In some aspects, a second pair of openings 606 may be disposed within the body 602 of the guide 600 and/or partially within the body 602 of the guide 600 and partially within the projection 604. As shown, the openings 606 are disposed within the projections 604 may have parallel central axes, whereas the second pair of openings 606 (disposed in the body 602) may have converging (or diverging) central axes. As shown, each of the openings 606 include substantially the same size and positioning relative to the projections 604 and the body 602. However, in some aspects the openings 606 may include alternate sizing and/or positioning. As shown, at least two of the openings 606 are positioned within the projections 604, however in some aspects one or more openings 606 may be positioned within the body 602 adjacent to the base of the projections 604. In some aspects, one or more pins may be placed within and/or through the openings 606 and into a bone (e.g., a tibia) so as to couple the guide 600 with the bone. In some aspects, the guide 600 may include a coupling element (e.g., a male or female dovetail component, etc.) extending from a portion of the guide 600 and configured to facilitate releasably coupling of the guide 600 with other instrumentation and components of a system, for example an ankle arthroplasty system.

The body 602 is further shown to include an opening 608 disposed in a central portion thereof. The opening 608 is shown to have a substantially arced geometry (e.g., curved, radiused, etc.) where an apex of the arced geometry is positioned laterally between the projections 604. The opening 608 may include a depth stop therein disposed partially between the top and bottom surfaces of the guide 600 such that an insert, for example the same as and/or similar to those shown and described subsequently herein, may be placed within and retained at a specific position/depth by the depth stop. Such an insert may include a plurality of openings, for example drill holes, through which holes may be drilled in the bone of a patient. The opening may also include one or more geometric features configured to accommodate such an insert with complimentary geometric features, with the geometric features configured to promote retention or nesting and stability of the insert within the opening 608. The opening 608 includes a substantially elongated, arcuate geometry and is positioned above one or more central windows defined on all sides by components of the guide 600, with the central windows configured to provide visibility of the anatomy (e.g., a joint) beneath the guide 600 when the guide 600 is coupled with the bone.

The guide 600 is further shown to include a pair of legs 612 extending downward (e.g., in a direction opposite that of the projections) from opposing lateral sides of the body 602. As shown, legs 612 extend at a substantially oblique angle relative to one another. Further, each of the legs 612 (at a point along a length thereof, for example the terminal end) define the greatest lateral dimension of the guide 600 on each side thereof. Each of the legs is shown to include a slot 616 disposed therein, wherein the slot 616 is defined laterally by each of the legs 612 and vertically in a downward direction by the body 602, but is undefined ((i.e., open) vertically in an upward direction where each of the slots is in fluid communication with the opening 608. Accordingly, each of the slots 616 may be configured to receive a cutting instrument therein (e.g., a reciprocating saw, sagittal saw, etc.) so as to facilitate the cutting/resection of the bone with which the guide 600 is coupled. As shown, the slots 616 are positioned substantially parallel to a central axis of each of the legs 612, however in some aspects the slots 616 may include alternate geometries.

The guide 600 is further shown to include an opening 610 disposed within a cross member extending laterally between the pair of legs 612. The opening 610 is disposed entirely within the cross member with the cross member being integrally connected to the pair of legs 612. The opening 610 may be configured to receive a surgical instrument therein, for example an alignment instrument such as an angelwing or other similar component. As shown, the opening 610 is a substantially horizontal opening and positioned in a plane parallel to a plane that is tangent to the arced geometry of the opening 608, however in some aspects the opening 610 may have alternate geometries. Additionally, the guide 600 is shown to include a pair of openings 620 positioned below the opening 610, where the pair of openings include substantially parallel central axes and are configured to receive a pin therein to facilitate coupling with a second bone of a patient (e.g., a talus).

The guide 600 is shown to include a frame member disposed within a central opening of the guide 600, with the central opening defined laterally by the legs 612, upward by the body 602 and downward by the cross member defining the opening 610. The frame member is shown to define an opening 618 therein, which as shown is configured as a substantially elongated, rectangular geometry and may be configured to receive therein an alignment instrument (e.g., an angelwing, etc.). The frame member is further shown to include at least one projection 614 on an outer surface thereof, extending into the central opening. As shown, the projections 614 are substantially triangular in shape and extend from the frame member adjacent opposite ends of the opening 618.

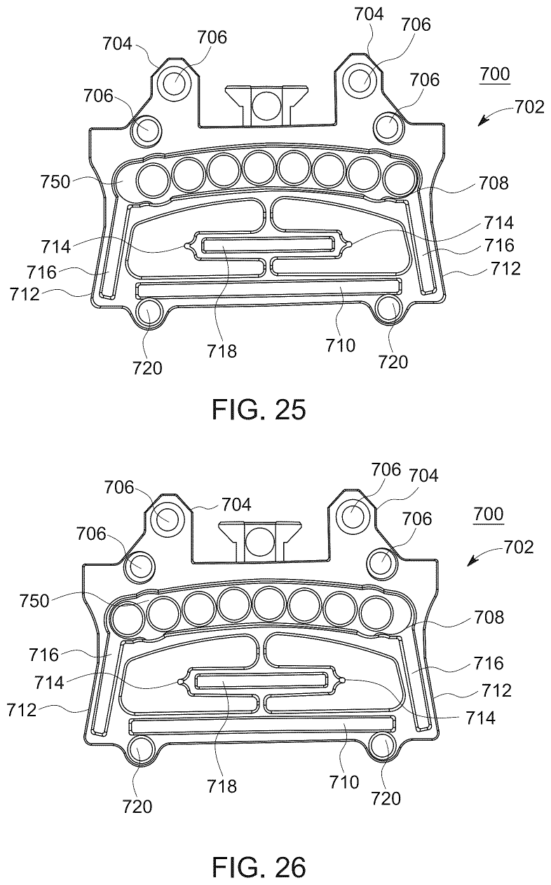

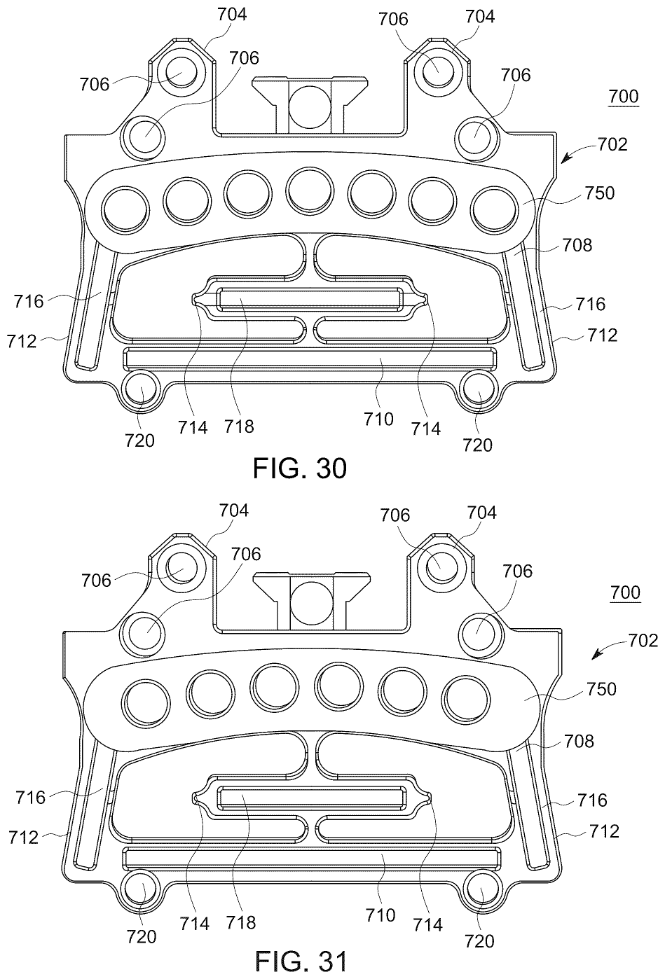

Referring now to FIGS. 25-31, a guide 700 and components which may be implemented in conjunction with the guide 700 are shown, according to an exemplary embodiment. The guide 700 is shown to include a body 702, with a pair of projections 704 extending upward from the body 702. As shown, the projections 704 extend upward from the body 702 substantially parallel to one another and, as shown, the projections 704 include substantially the same geometry (although in some aspects the projections 704 may include alternate geometries from that shown and/or from one another). Each of the projections 704 are shown to include at least one opening 706 (e.g., a bore, etc.), where at least one of the openings 706 is centrally positioned relative to the projection 704 and extends from a top surface of the guide through to the bottom surface. In some aspects, one or more of the openings 706 may be biased toward a terminal end of the projections 704. In some aspects, a second pair of openings 706 may be disposed within the body 702 of the guide 700 and/or partially within the body 702 of the guide 700 and partially within the projection 704. As shown, the openings 706 disposed within the projections 704 may have parallel central axes, whereas the second pair of openings 706 (disposed in the body 702) may have converging (or diverging) central axes. As shown, each of the openings 706 include substantially the same size and positioning relative to the projections 704 and the body 702. However, in some aspects the openings 706 may include alternate sizing and/or positioning. As shown, at least two of the openings 706 are positioned within the projections 704, however in some aspects one or more openings 706 may be positioned within the body 702 adjacent to the base of the projections 704. In some aspects, one or more pins may be placed within and/or through the openings 706 and into a bone (e.g., a tibia) so as to couple the guide 700 with the bone. In some aspects, the guide 700 may include a coupling element (e.g., a male or female dovetail component, etc.) extending from a portion of the guide 700 and configured to facilitate releasably coupling of the guide 700 with other instrumentation and components of a system, for example an ankle arthroplasty system.

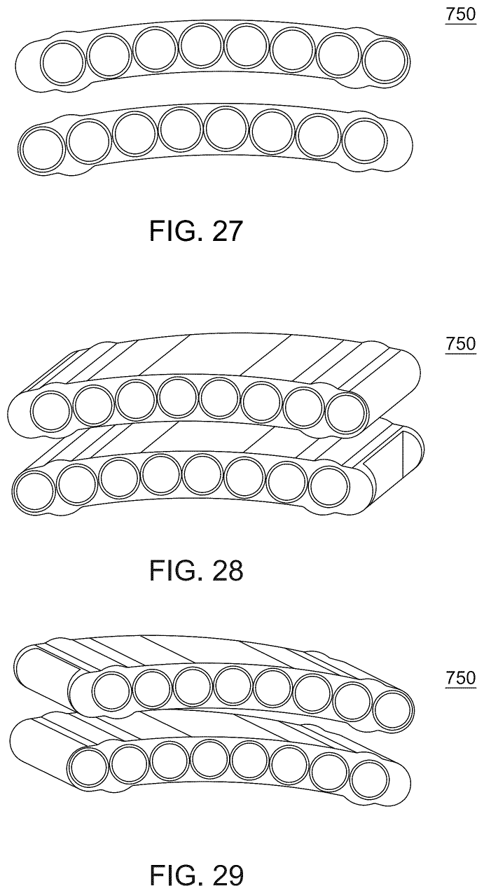

The body 702 is further shown to include an opening 708 disposed in a central portion thereof. The opening 708 is shown to have a substantially arced geometry (e.g., curved, radiused, etc.) where an apex of the arced geometry is positioned laterally between the projections 704. The opening 708 may include a depth stop therein disposed partially between the top and bottom surfaces of the guide 700 such that an insert, for example an insert 750 (See FIGS. 27-29), may be placed within the opening and retained at a specific position/depth by the depth stop. The insert 750 is shown in FIGS. 27-29 to include a plurality of openings, (shown as drill holes), through which holes may be drilled in the bone of a patient. The opening 708 may also include one or more geometric features configured to accommodate the insert 750 with complimentary geometric features, with the geometric features configured to promote retention/nesting and stability of the insert 750 within the opening 708. The opening 708 includes a substantially elongated, arcuate geometry and is positioned above one or more central windows defined on all sides by components of the guide 700, with the central windows configured to provide visibility of the anatomy (e.g., a joint) beneath the guide 700 when the guide 700 is coupled to the bone.

The guide 700 is further shown to include a pair of legs 712 extending downward (e.g., in a direction opposite that of the projections) from opposing lateral sides of the body 702. As shown, legs 712 extend at a substantially oblique angle relative to one another. Further, each of the legs 712 (at a point along a length thereof, for example the terminal end) define the greatest lateral dimension of the guide 700 on each side thereof. Each of the legs 712 is shown to include a slot 716 disposed therein, wherein the slot 716 is defined laterally by each of the legs 712 and vertically in a downward direction by the body 702, but is undefined (i.e., open) vertically in an upward direction where each of the slots 716 is in fluid communication with the opening 708. Accordingly, each of the slots 716 may be configured to receive a cutting instrument therein (e.g., a reciprocating saw, sagittal saw, etc.) so as to facilitate the cutting/resection of the bone to which the guide 700 is attached. As shown, the slots 716 are positioned substantially parallel to a central axis of each of the pair of legs 712, however in some aspects the slots 716 may include alternate geometries.

The guide 700 is further shown to include an opening 710 disposed within a cross member extending laterally between the pair of legs 712. The opening 710 is disposed entirely within the cross member with the cross member being integrally attached to the pair of legs 712. The opening 710 may be configured to receive a surgical instrument therein, for example an alignment instrument such as an angelwing or other similar component. As shown, the opening 710 is a substantially horizontal opening and positioned in a plane parallel to a plane that is tangent to the arced geometry of the opening 708, however in some aspects, the opening 710 may have alternate geometries. Additionally, the guide 700 is shown to include a pair of openings 720 positioned below the opening 710, where the pair of openings 720 include substantially parallel central axes and are configured to receive a pin therein to facilitate coupling with a second bone of a patient (e.g., a talus).

The guide 700 is shown to include a frame member disposed within a central opening of the guide 700, with the central opening 710 defined laterally by the legs 712, upward by the body 702 and downward by the cross member. The frame member is shown to define an opening 718 therein, which as shown is configured as a substantially elongated, rectangular geometry and may be configured to receive therein an alignment instrument (e.g., an angelwing, etc.). The frame member is further shown to include at least one projection 714 on an outer surface thereof, extending into the central opening 710. As shown, the projections 714 are substantially triangular in shape and extend from the frame member adjacent opposite ends of the opening 718.

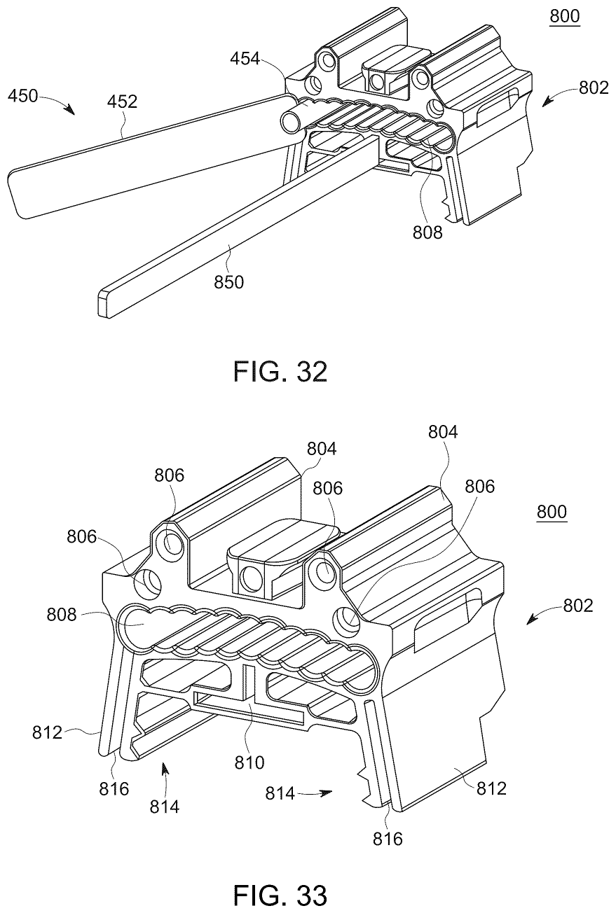

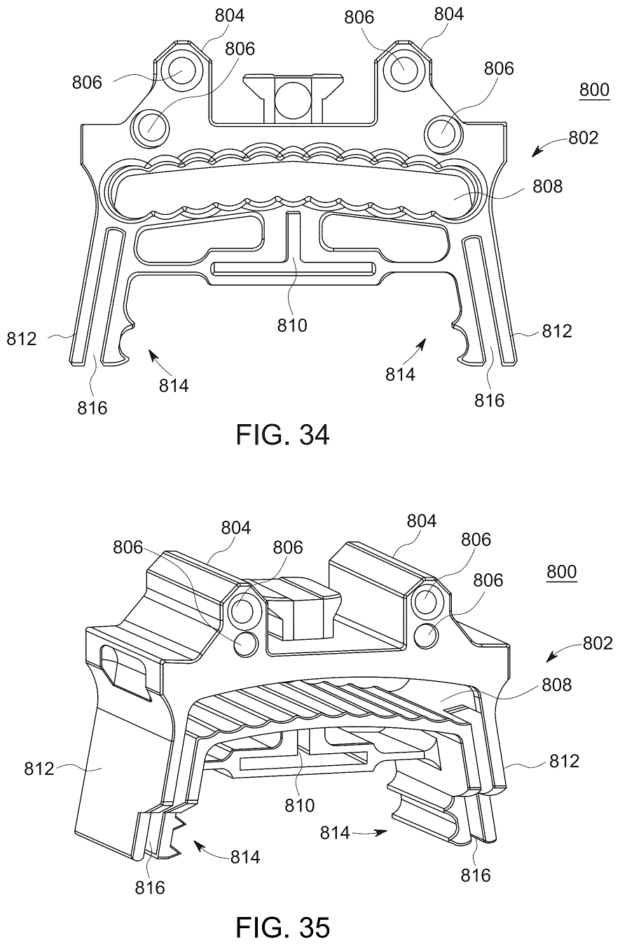

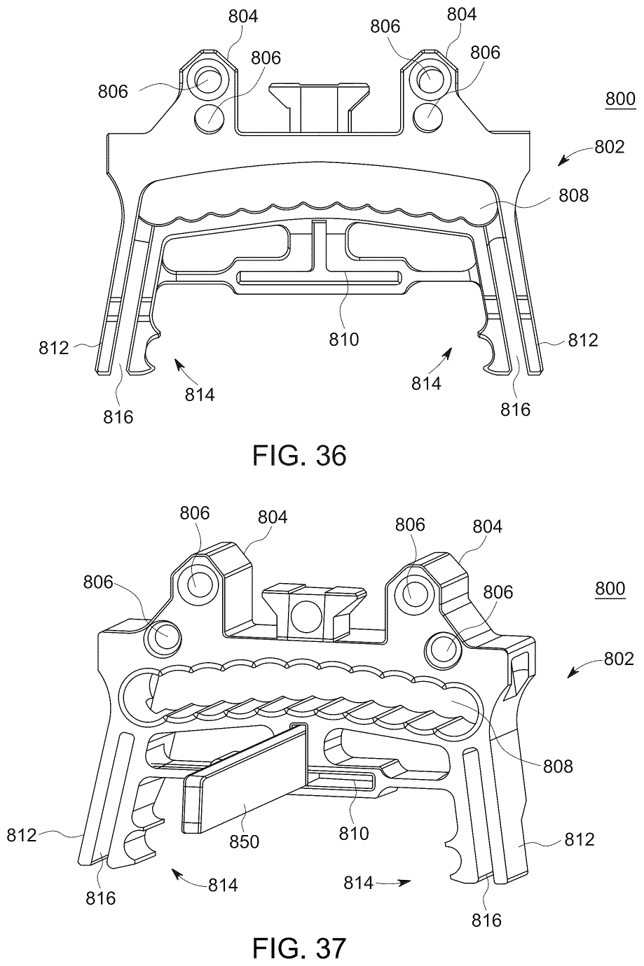

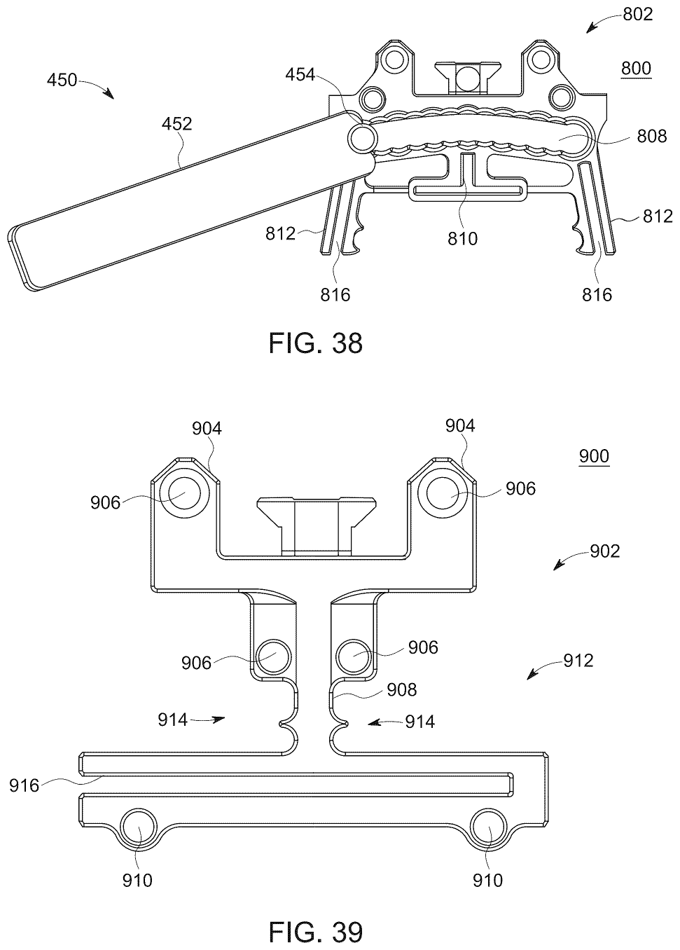

Referring now to FIGS. 32-38 and FIG. 42, a guide 800 and components which may be implemented in conjunction with the guide 800 are shown, according to an exemplary embodiment. The guide 800 is shown to include a body 802, with a pair of projections 804 extending upward from the body 802. As shown, the projections 804 extend upward from the body 802 substantially parallel to one another and, as shown, the projections 804 include substantially the same geometry (although in some aspects the projections 804 may include alternate geometries from that shown and/or from one another). Each of the projections 804 are shown to include at least one opening 806 (e.g., a bore, etc.), where at least one of the openings 806 is centrally positioned relative to the projection 804 and extending from a top surface of the guide through to the bottom surface. In some aspects, one or more of the openings 806 may be biased toward a terminal end of the projections 804. In some aspects, a second pair of openings 806 may be disposed within the body 802 of the guide 800 and/or partially within the body 802 of the guide 800 and partially within the projection 804. As shown, the openings 806 disposed within the projections 804 may have parallel central axes, whereas the second pair of openings 806 (disposed in the body 802) may have converging (or diverging) central axes. As shown, each of the openings 806 include substantially the same size and positioning relative to the projections 804 and the body 802. However, in some aspects the openings 806 may include alternate sizing and/or positioning. As shown, at least two of the openings 806 are positioned within the projections 804, however in some aspects one or more openings 806 may be positioned within the body 802 adjacent to the base of the projections 804. In some aspects, one or more pins may be placed within and/or through the openings 806 and into a bone (e.g., a tibia) so as to couple the guide 800 with the bone. In some aspects, the guide 800 may include a coupling element (e.g., a male or female dovetail component, etc.) extending from a portion of the guide 800 and configured to facilitate releasably coupling of the guide 800 with other instrumentation and components of a system, for example an ankle arthroplasty system.

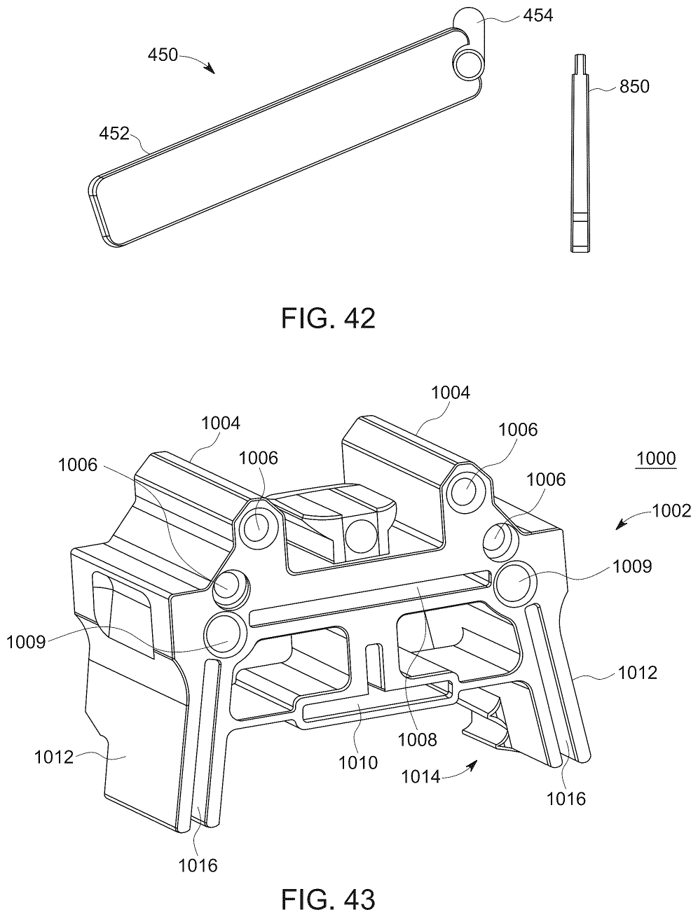

The body 802 is further shown to include an opening 808 disposed in a central portion thereof. The opening 808 is shown to have a substantially arced geometry (e.g., curved, radiused, etc.) where an apex of the arced geometry is positioned laterally between the projections 804. Further, the opening 808 is shown to include a plurality of scallops disposed within, where the scallops extend from at least one of upper, lower, and lateral sides of the opening 808, with each of the scallops defining at least a portion (but not necessarily an entirety) of a cylindrical geometry (or other similar geometry) extending from the top surface of the guide 800 at least partially through to the bottom surface (e.g., through the depth of the opening 808). Accordingly, a drill guide 450 as shown in FIGS. 32 and 42, includes a handle 452 and a shaft 454 that may be manipulated such that the shaft 454 is inserted at least partially within one or more of the scallops of the opening 808. As shown, the shaft 454 is configured to have a complimentary size and geometry to the scallops (e.g., a cylindrical drill guide) to facilitate placement of the shaft 454 within the scallops, and thus guide a drill therein and therethrough so as to drill into the bone with which the guide 800 is coupled. The opening 808 is positioned above at least one central window defined on all sides by components of the guide 800, with the central window configured to provide visibility of the anatomy (e.g., a joint) beneath the guide 800 when the guide 800 is coupled with the bone.

The guide 800 is further shown to include a pair of legs 812 extending downward (e.g., in a direction opposite that of the projections) from opposing lateral sides of the body 802. As shown, legs 812 extend at a substantially oblique angle relative to one another. Further, each of the legs 812 (at a point along a length thereof, for example the terminal end) defines the greatest lateral dimension of the guide 800 on each side thereof. Each of the legs 812 are shown to include at least one projection 814 on an inner surface thereof. As shown, the at least one of the projections 814 is substantially triangular in shape and points toward the central opening positioned between the legs 812 and defined laterally by the legs 812. Each of the legs is shown to include a slot 816 disposed therein, wherein the slot 816 is defined laterally by each of the legs 812 and vertically in an upward direction by the body 802, but is undefined (i.e., open) vertically in a lower direction. Accordingly, each of the slots 816 may be configured to receive a cutting instrument therein (e.g., a reciprocating saw, sagittal saw, etc.) so as to facilitate the cutting/resection of the bone to which the guide 800 is coupled. As shown, the slots 816 are positioned substantially parallel to a central axis of each of the legs 812, however in some aspects the slots 816 may include alternate geometries.

The guide 800 is further shown to include an opening 810 disposed within a cross member extending laterally between the pair of legs 812 and downward from the body 802 at a point below the opening 808. The opening 810 is disposed entirely within the cross member. The cross member is integral with the pair of legs 812. The opening 810 may be configured to receive a surgical instrument therein, for example an alignment instrument 850 as shown in FIGS. 32, 37 and 42, or an angelwing or other similar component. As shown, the opening 810 includes both horizontal and vertical rectangular geometries, collectively forming an upside-down “T” shaped opening. The horizontal rectangular portion is positioned in a plane parallel to a plane that is tangent to the arced geometry of the opening 808, however in some aspects the opening 810 may have alternate geometries.

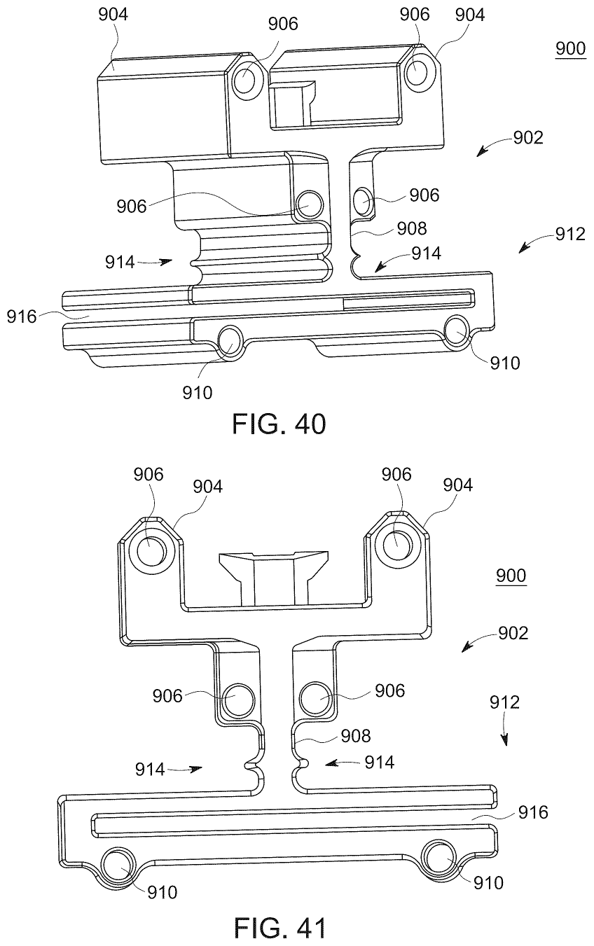

Referring now to FIGS. 39-41, a guide 900 is shown, according to an exemplary embodiment. The guide 900 is shown to include a body 902, with a pair of projections 904 extending upwardly from the body 902. As shown, the projections 904 extend upward from the body 902 substantially parallel to one another and, as shown, the projections 904 include substantially the same geometry (although in some aspects the projections 904 may include alternate geometries from that shown and/or from one another). Each of the projections 904 are shown to include at least one opening 906 (e.g., a bore, etc.), where at least one of the openings 906 is centrally positioned relative to the projection 904 and extending from a top surface of the guide through to the bottom surface. In some aspects, one or more of the openings 906 may be biased toward a terminal end of the projections 904. As shown, a second pair of openings 906 is disposed within the body 902 of the guide 900. As shown, the openings 906 disposed within the projections 904 may have parallel central axes, whereas the second pair of openings 906 (disposed in the body 902) are shown to have converging (but may alternatively have diverging) central axes. As shown, each pair of openings 906 include substantially the same size and positioning relative to the projections 904 and the body 902. However, in some aspects the openings 906 may include alternate sizing and/or positioning. As shown, at least two of the openings 906 are positioned within the projections 904, however in some aspects one or more openings 906 may be positioned within the body 902 adjacent to the base of the projections 904. In some aspects, one or more pins may be placed within and/or through the openings 906 and into a bone (e.g., a tibia) so as to couple the guide 900 with the bone. In some aspects, the guide 900 may include a coupling element (e.g., a male or female dovetail component, etc.) extending from a portion of the guide 900 and configured to facilitate releasably coupling of the guide 900 with other instrumentation and components of a system, for example an ankle arthroplasty system.

The guide 900 is further shown to include a shaft 908 extending downward from the body 902 to a frame 912 disposed substantially below the body 902. As shown, the body 902, shaft 908, and frame 912 are integral with one another with the shaft 908 extending from both the body 902 and the frame 912 at a substantially orthogonal angle from surfaces of the respective components. The shaft 908 is shown to include at least one projection 914 on an outer surface thereof. As shown, at least one of the projections 914 is substantially triangular in shape and points outward from a surface of the shaft 908 at a substantially orthogonal direction. The frame 912 is shown to include a pair of openings 910 on a lower portion thereof, with the openings configured to facilitate coupling of the frame 912 with a second bone of a patient (for example, a talus). As shown, the openings 910 are positioned below a slot 916 disposed within the frame 912, where the slot is defined in vertical directions (upward and downward) and on a lateral side by the frame 912. Further, the slot 916 is undefined (i.e., open) on one side (of a rectangular geometry, as shown) so as to facilitate the insertion and removal of a cutting instrument, for example a reciprocating or sagittal saw. In some aspects the guide 900 may be reversible, such that the guide 900 may be placed over pins received in the openings 906, 910, a cut made within the slot 916, then the guide 900 removed, flipped 180 degrees, and repositioned with the pins received in the openings 906, 910 and the open side of the slot 916 facing an opposite direction from the initial position so as to facilitate an extension of the first cut or guidance of a second cut.

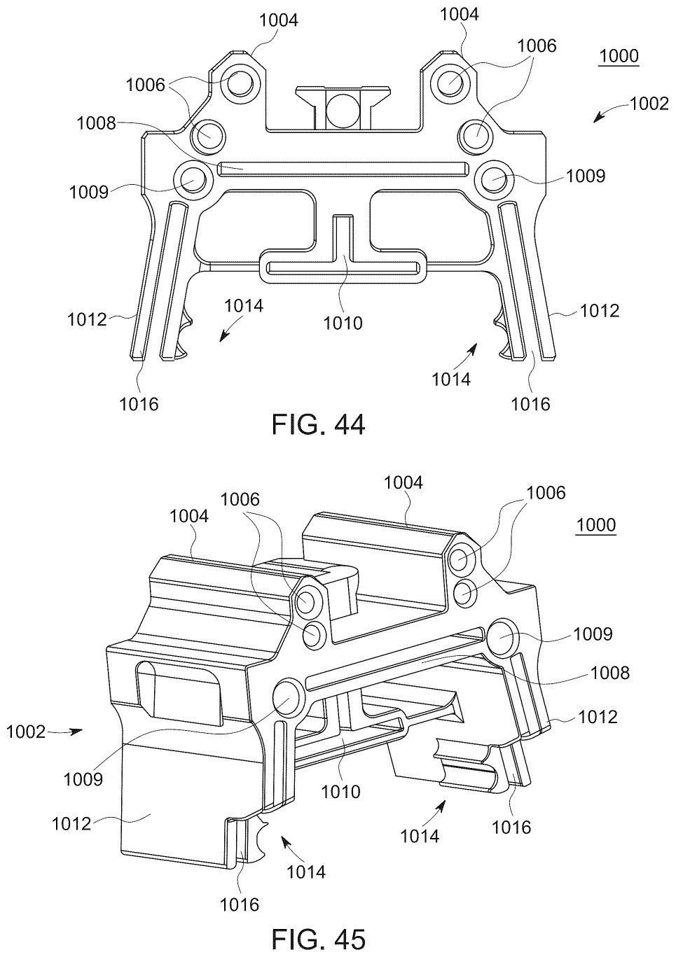

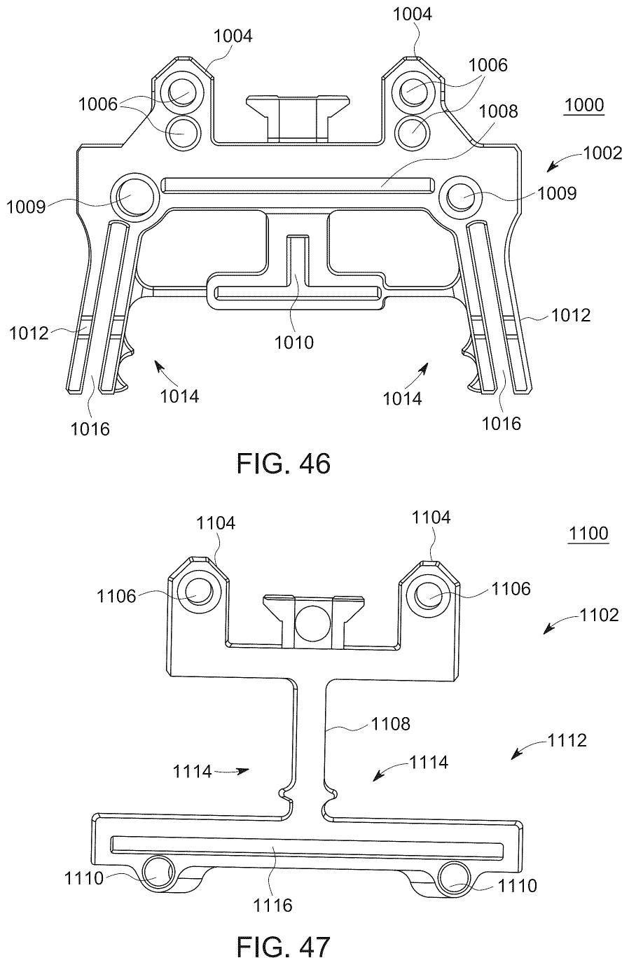

Referring now to FIGS. 43-46, a guide 1000 is shown, according to an exemplary embodiment. The guide 1000 is shown to include a body 1002, with a pair of projections 1004 extending upward from the body 1002. As shown, the projections 1004 extend upward from the body 1002 substantially parallel to one another and, as shown, the projections 1004 include substantially the same geometry (although in some aspects the projections 1004 may include alternate geometries from that shown and/or from one another). Each of the projections 1004 are shown to include at least one opening 1006 (e.g., a bore, etc.), where at least one of the openings 1006 is centrally positioned relative to the projection 1004 and extending from a top surface of the guide through to the bottom surface. In some aspects, one or more of the openings 1006 may be biased toward a terminal end of the projections 1004. In some aspects, a second pair of openings 1006 may be disposed within the body 1002 of the guide 1000 and/or partially within the body 1002 of the guide 1000 and partially within the projection 1004. As shown, the openings 1006 disposed within the projections 1004 may have parallel central axes, whereas the second pair of openings 1006 (disposed in the body 1002) may have converging (or diverging) central axes. As shown, each of the openings 1006 include substantially the same size and positioning relative to the projections 1004 and the body 1002. However, in some aspects the openings 1006 may include alternate sizing and/or positioning. As shown, at least two of the openings 1006 are positioned within the projections 1004, however in some aspects one or more openings 1006 may be positioned within the body 1002 adjacent to the base of the projections 1004. In some aspects, one or more pins may be placed within and/or through the openings 1006 and into a bone (e.g., a tibia) so as to couple the guide 1000 with the bone. In some aspects, the guide 1000 may include a coupling element (e.g., a male or female dovetail component, etc.) extending from a portion of the guide 1000 and configured to facilitate releasably coupling the guide 1000 with other instrumentation and components of a system, for example an ankle arthroplasty system.

The body 1002 is further shown to include an opening 1008 disposed in a central portion thereof. The opening 1008 includes a substantially elongated, rectangular geometry and is positioned above at least one central window defined on all sides by components of the guide 1000, with the central window configured to provide visibility of the anatomy (e.g., a joint) beneath the guide 1000 when the guide 1000 is coupled with the bone. The body 1002 is also shown to include a pair of openings 1009 positioned on opposite ends of and adjacent to the opening 1008. As shown, the openings 1009 include a substantially cylindrical geometry where central axes of the openings 1009 are substantially parallel to one another. Accordingly, portions of a tibial resection (with the guide 1000 coupled with the tibia of a patient) may be performed by drilling holes in the tibia through each of the openings 1009 and cutting (with a reciprocating or sagittal saw) into the tibia through and along the opening 1008.

The guide 1000 is further shown to include a pair of legs 1012 extending downward (e.g., in a direction opposite that of the projections) from opposing lateral sides of the body 1002. As shown, the pair of legs 1012 extend from a point just below and adjacent to the openings 1009 at a substantially oblique angle relative to one another. Further, each of the pair of legs 1012 (at a point along a length thereof, for example the terminal end) defines the greatest lateral dimension of the guide 1000 on each side thereof. Each of the pair of legs 1012 are shown to include at least one projection 1014 on an inner surface thereof. As shown, at least one of the projections 1014 is substantially triangular in shape and points toward the central opening positioned between the legs 1012 and defined laterally by the pair of legs 1012. Each of the pair of legs is shown to include a slot 1016 disposed therein, wherein the slot 1016 is defined laterally by each of the legs 1012 and vertically in an upward direction by the body 1002, but is undefined vertically (i.e., open) in a lower direction. Accordingly, each of the slots 1016 may be configured to receive a cutting instrument therein (e.g., a reciprocating saw, sagittal saw, etc.) so as to facilitate the cutting/resection of the bone with which the guide 1000 is coupled. As shown, the slots 1016 are positioned substantially parallel to a central axis of each of the pair of legs 1012, however in some aspects the slots 1016 may include alternate geometries.

The guide 1000 is further shown to include an opening 1010 disposed within a cross member extending laterally between the pair of legs 1012 and downward from the body 1002 at a point below the opening 1008. The opening 1010 is disposed entirely within the cross member. The cross member being integral with the pair of legs 1012. The opening 1010 may be configured to receive a surgical instrument therein, for example an alignment instrument 850 as shown in FIGS. 32, 37 and 42, or an angelwing or other similar component. As shown, the opening 1010 includes both horizontal and vertical rectangular geometries, collectively forming an upside-down “T” shaped opening. The horizontal rectangular portion is positioned in a plane parallel to a plane that is tangent to the arced geometry of the opening 1008, however in some aspects the opening 1010 may have alternate geometries.

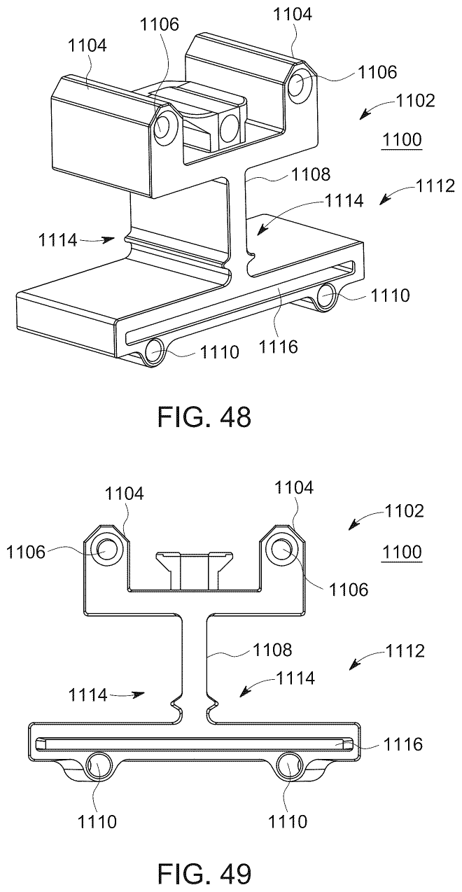

Referring now to FIGS. 47-49, a guide 1100 is shown, according to an exemplary embodiment. The guide 1100 is shown to include a body 1102, with a pair of projections 1104 extending upwardly from the body 1102. As shown, the projections 1104 extend upward from the body 1102 substantially parallel to one another and, as shown, the projections 1104 include substantially the same geometry (although in some aspects the projections 1104 may include alternate geometries from that shown and/or from one another). Each of the projections 1104 are shown to include at least one opening 1106 (e.g., a bore, etc.), where at least one of the openings 1106 is centrally positioned relative to the projection 1104 and extending from a top surface of the guide through to the bottom surface. In some aspects, one or more of the openings 1106 may be biased toward a terminal end of the projections 1104. As shown, a second pair of openings 1106 is disposed within the body 1102 of the guide 1100. As shown, the openings 1106 disposed within the projections 1104 may have parallel central axes, whereas the second pair of openings 1106 (disposed in the body 1102) are shown to have converging (but may alternatively have diverging) central axes. As shown, each pair of openings 1106 include substantially the same size and positioning relative to the projections 1104 and the body 1102. However, in some aspects the openings 1106 may include alternate sizing and/or positioning. As shown, at least two of the openings 1106 are positioned within the projections 1104, however in some aspects one or more openings 1106 may be positioned within the body 1102 adjacent to the base of the projections 1104. In some aspects, one or more pins may be placed within and/or through the openings 1106 and into a bone (e.g., a tibia) so as to couple the guide 1100 with the bone. In some aspects, the guide 1100 may include a coupling element (e.g., a male or female dovetail component, etc.) extending from a portion of the guide 1100 and configured to facilitate releasably coupling the guide 1100 with other instrumentation and components of a system, for example an ankle arthroplasty system.

The guide 1100 is further shown to include a shaft 1108 extending downward from the body 1102 to a frame 1112 disposed substantially below the body 1102. As shown, the body 1102, shaft 1108, and frame 1112 are integral with one another, with the shaft 1108 extending from both the body 1102 and the frame 1112 at a substantially orthogonal angle from the surfaces of the respective components. The shaft 1108 is shown to include at least one projection 1114 on an outer surface thereof. As shown, at least one of the projections 1114 is substantially triangular in shape and points outward from a surface of the shaft 1108 at a substantially orthogonal direction. The frame 1112 is shown to include a pair of openings 1110 on a lower portion thereof, with the openings configured to facilitate coupling of the frame 1112 with a second bone of a patient (for example, a talus). The openings 110 are shown to include central axes that are substantially converging (but may alternatively be diverging). As shown, the openings 1110 are positioned below a slot 1116 disposed within the frame 1112, where the slot is defined in vertical directions (upward and downward) and on a lateral side by the frame 1112. Further, the slot 1116 is undefined (i.e., open) on one side (of a rectangular geometry, as shown) so as to facilitate the insertion and removal of a cutting instrument, for example a reciprocating or sagittal saw.

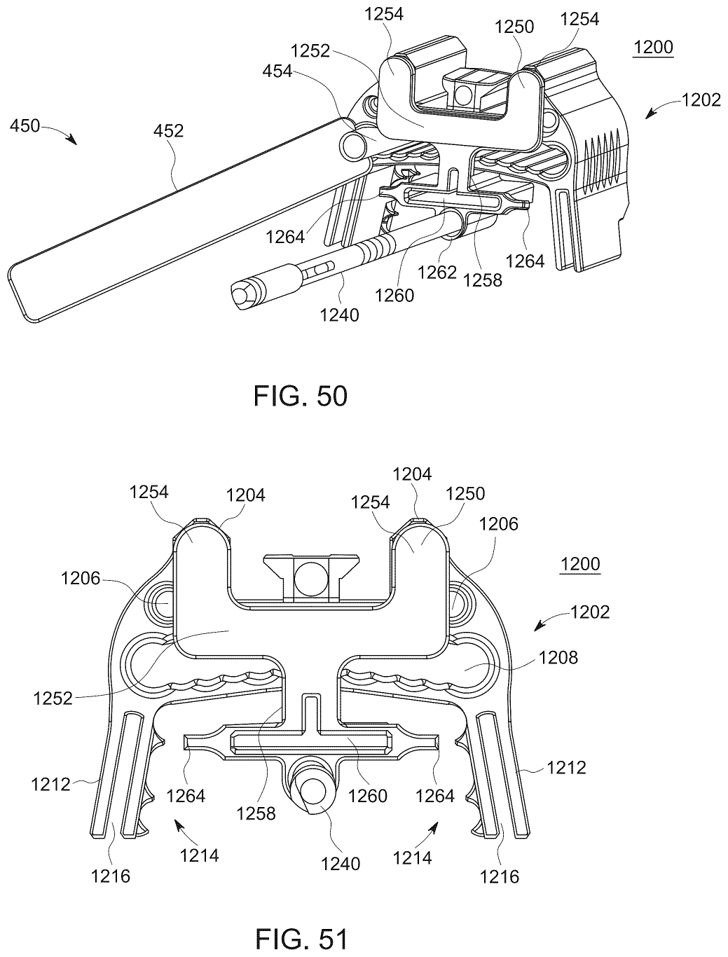

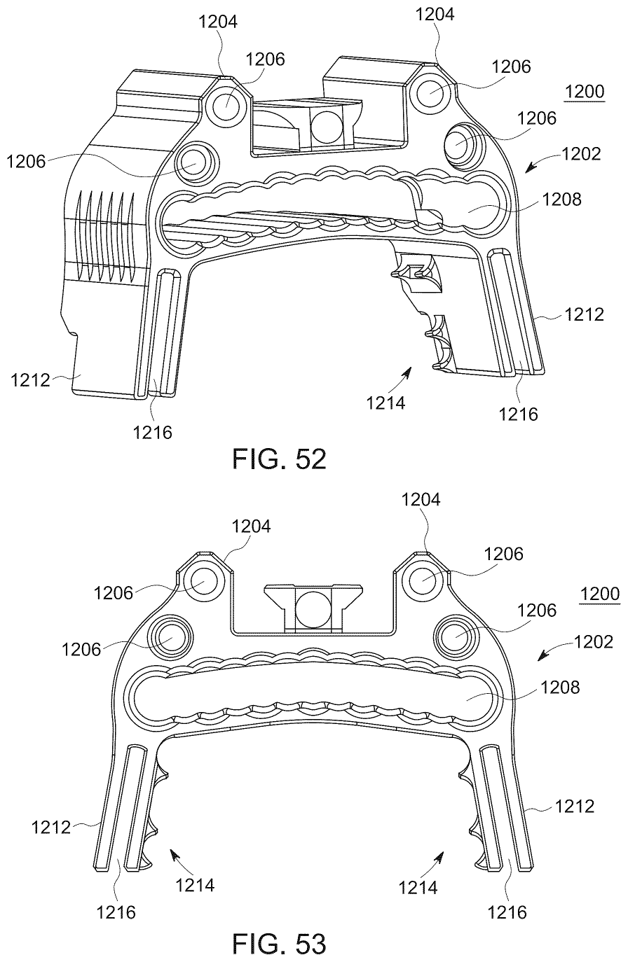

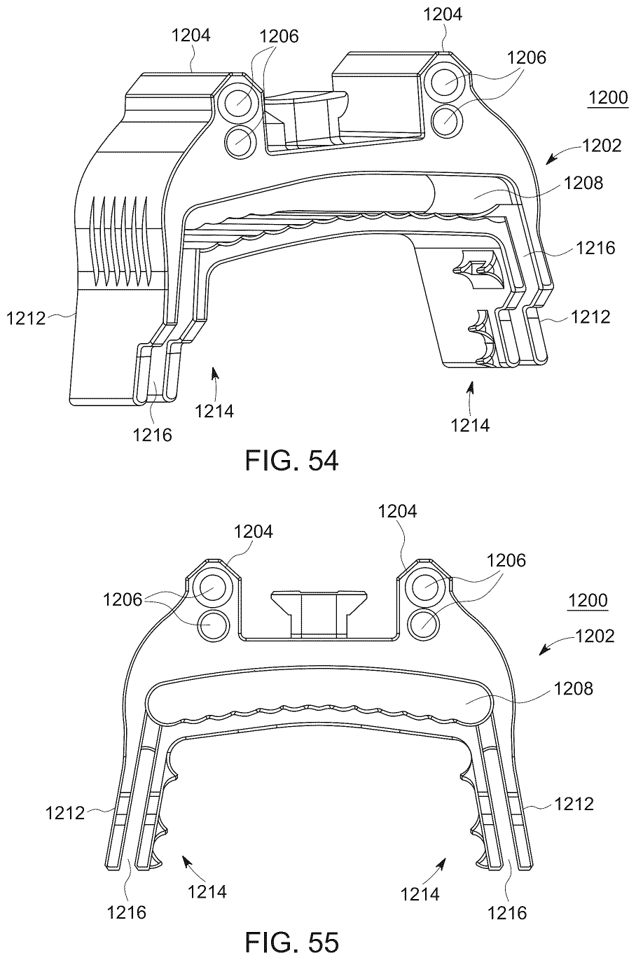

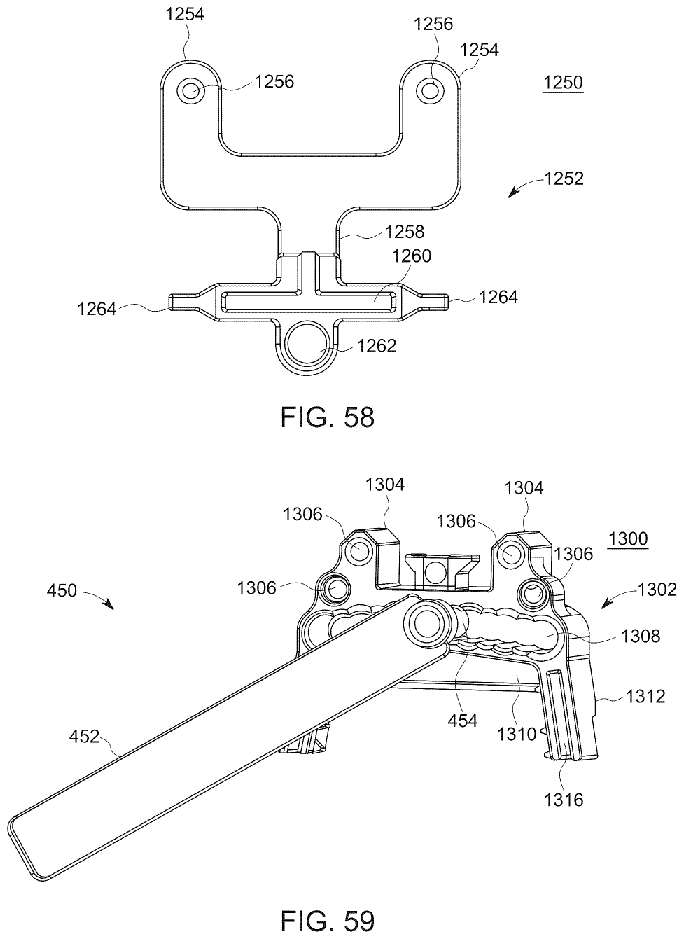

Referring now to FIGS. 50-58, a guide 1200 and components which may be implemented in conjunction with the guide 1200 are shown, according to an exemplary embodiment. The guide 1200 is shown to include a body 1202, with a pair of projections 1204 extending upwardly from the body 1202. As shown, the projections 1204 extend upward from the body 1202 substantially parallel to one another and, as shown, the projections 1204 include substantially the same geometry (although in some aspects the projections 1204 may include alternate geometries from that shown and/or from one another). Each of the projections 1204 are shown to include at least one opening 1206 (e.g., a bore, etc.), where at least one of the openings 1206 is centrally positioned relative to the projection 1204 and extending from a top surface of the guide through to the bottom surface. In some aspects, one or more of the openings 1206 may be biased toward a terminal end of the projections 1204. In some aspects, a second pair of openings 1206 may be disposed within the body 1202 of the guide 1200 and/or partially within the body 1202 of the guide 1200 and partially within the projection 1204. As shown, the openings 1206 disposed within the projections 1204 may have parallel central axes, whereas the second pair of openings 1206 (disposed in the body 1202) may have converging (or diverging) central axes. As shown, each of the openings 1206 include substantially the same size and positioning relative to the projections 1204 and the body 1202. However, in some aspects the openings 1206 may include alternate sizing and/or positioning. As shown, at least two of the openings 1206 are positioned within the projections 1204, however in some aspects one or more openings 1206 may be positioned within the body 1202 adjacent to the base of the projections 1204. In some aspects, one or more pins may be placed within and/or through the openings 1206 and into a bone (e.g., a tibia) so as to couple the guide 1200 with the bone. In some aspects, the guide 1200 may include a coupling element (e.g., a male or female dovetail component, etc.) extending from a portion of the guide 1200 and configured to facilitate releasably coupling of the guide 1200 with other instrumentation and components of a system, for example an ankle arthroplasty system.

The body 1202 is further shown to include an opening 1208 disposed in a central portion thereof. The opening 1208 is shown to have a substantially arced geometry (e.g., curved, radiused, etc.) where an apex of the arced geometry is positioned laterally between the projections 1204. Further, the opening 1208 is shown to include a plurality of scallops disposed within, where the scallops extend from at least one of upper, lower, and lateral sides of the opening 1208, with each of the scallops defining at least a portion (but not necessarily an entirety) of a cylindrical geometry (or other similar geometry) extending from the top surface of the guide 1200 at least partially through to the bottom surface (e.g., through the depth of the opening 1208). Accordingly, the drill guide 450 as shown in FIGS. 32 and 42, may be manipulated such that the shaft 454 is inserted at least partially within one or more of the scallops of the opening 1208. The opening 1208 is positioned above at least one central window defined on multiple sides by components of the guide 1200, with the central window configured to provide visibility of the anatomy (e.g., a joint) beneath the guide 1200 when the guide 1200 is coupled with a bone.

The guide 1200 is further shown to include a pair of legs 1212 extending downward (e.g., in a direction opposite that of the projections) from opposing lateral sides of the body 1202. As shown, the pair of legs 1212 extend from a point just below and adjacent to the openings 1009 at a substantially oblique angle relative to one another. Further, each of the pair of legs 1212 (at a point along a length thereof, for example the terminal end) defines the greatest lateral dimension of the guide 1200 on each side thereof. Each of the pair of legs 1212 are shown to include at least one projection 1214 on an inner surface thereof. As shown, at least one of the projections 1214 is substantially triangular in shape and points toward the central opening positioned between the pair of legs 1212 and defined laterally by the legs 1212. Each of the pair of legs is shown to include a slot 1216 disposed therein, wherein the slot 1216 is defined laterally by each of the legs 1212 and vertically in an upward direction by the body 1202, but is undefined (i.e., open) vertically in a lower direction. Accordingly, each of the slots 1216 may be configured to receive a cutting instrument therein (e.g., a reciprocating saw, sagittal saw, etc.) so as to facilitate the cutting/resection of the bone with which the guide 1200 is coupled. As shown, the slots 1216 are positioned substantially parallel to a central axis of each of the pair of legs 1212, however in some aspects the slots 1216 may include alternate geometries.

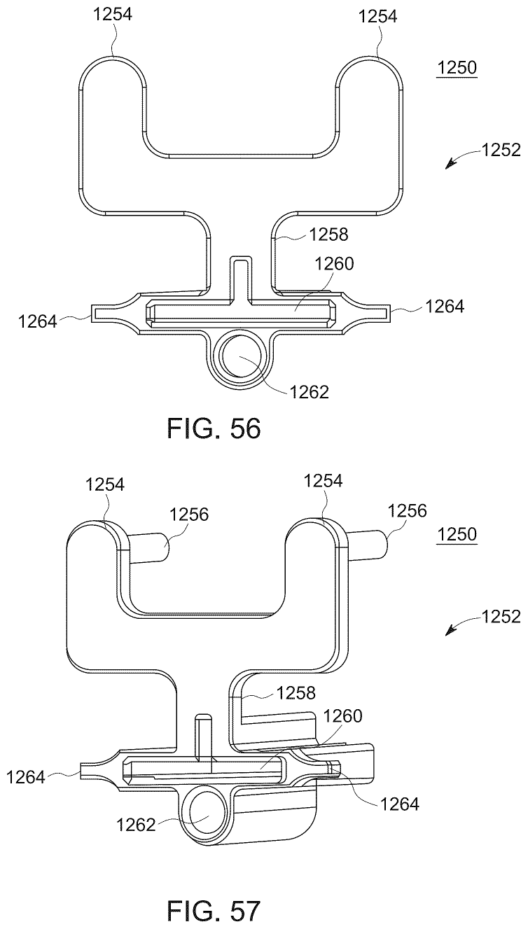

The guide 1200 is shown to be releasably couplable with an instrument 1250 and/or releasably couple with one or more instruments, for example the instrument 1240 shown in at least FIGS. 50 and 51. The instrument 1250 is shown in FIGS. 56-58 to include a pair of projections 1254 extending upwardly from a body 1252 substantially parallel to one another. Each of the projections 1254 are shown to include an extension 1256 extending substantially perpendicularly from the bottom surface of the projections 1254, where the extensions 1256 as shown to include a substantially cylindrical geometry with dimensions configured to facilitate reception within at least two of the openings 1206 of the guide 1200. The instrument 1250 is further shown to include a shaft 1258 extending downward from the body 1252 which includes an opening 1260 therein. As shown, the opening 1260 includes both horizontal and vertical rectangular geometries, collectively forming an upside-down “T” shaped opening and configured to receive at least a portion of an instrument therein, for example the instrument 850. Positioned on opposing sides of the shaft 1258 and adjacent the opening 1260 are a pair of protrusions 1264, which extend laterally from the shaft 1258. The shaft 1258 further includes an opening 1262 positioned below the opening 1260 which, as shown, includes a cylindrical geometry and is configured to releasably couple with the instrument 1240 as shown in FIGS. 50-51.

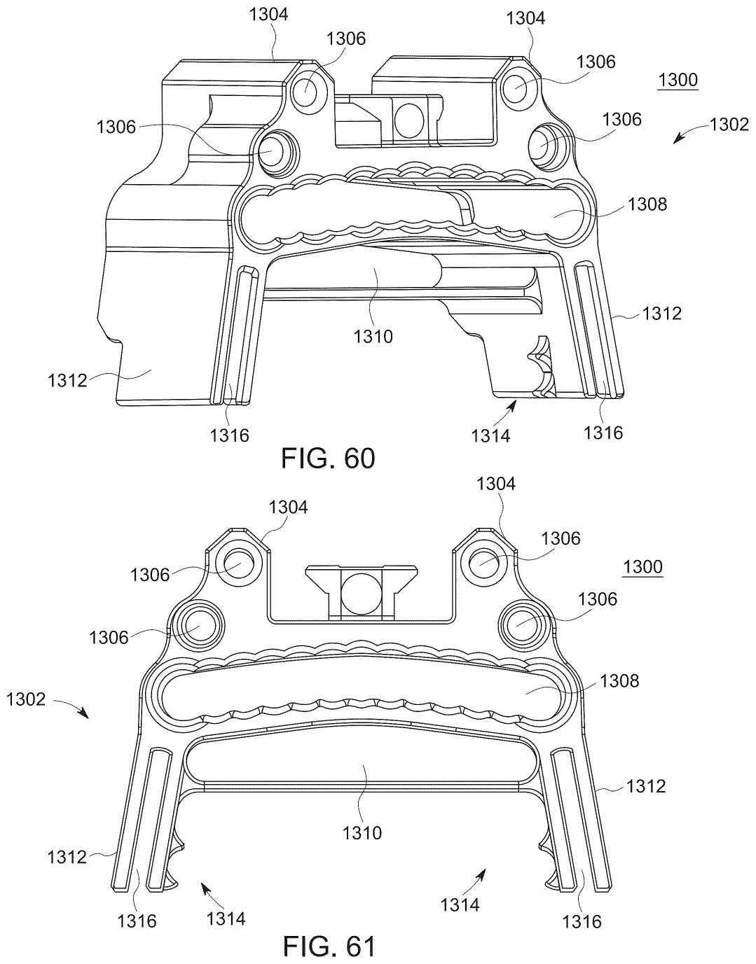

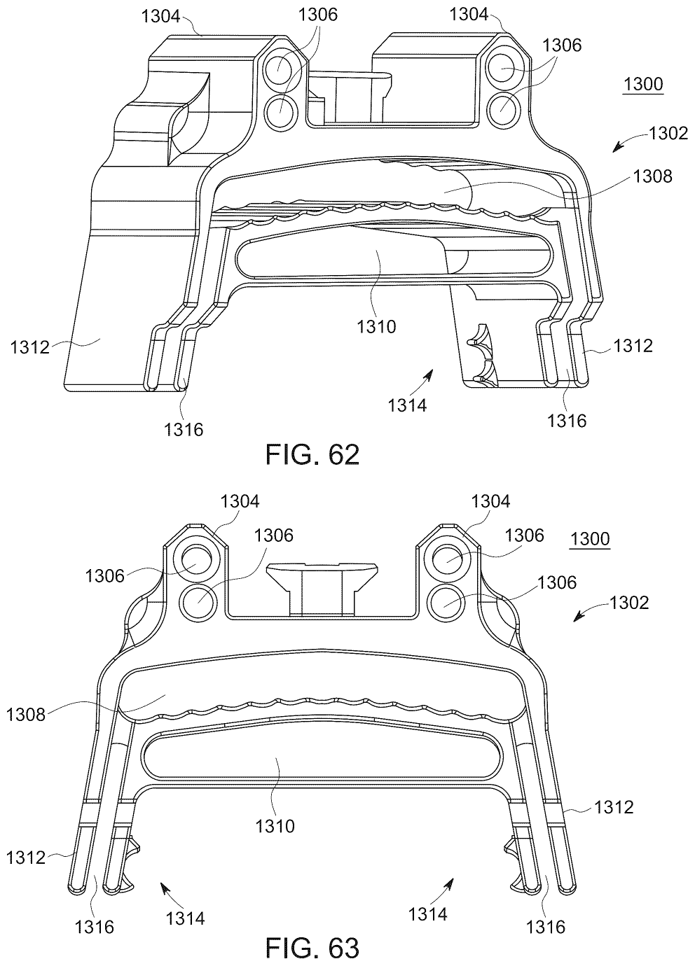

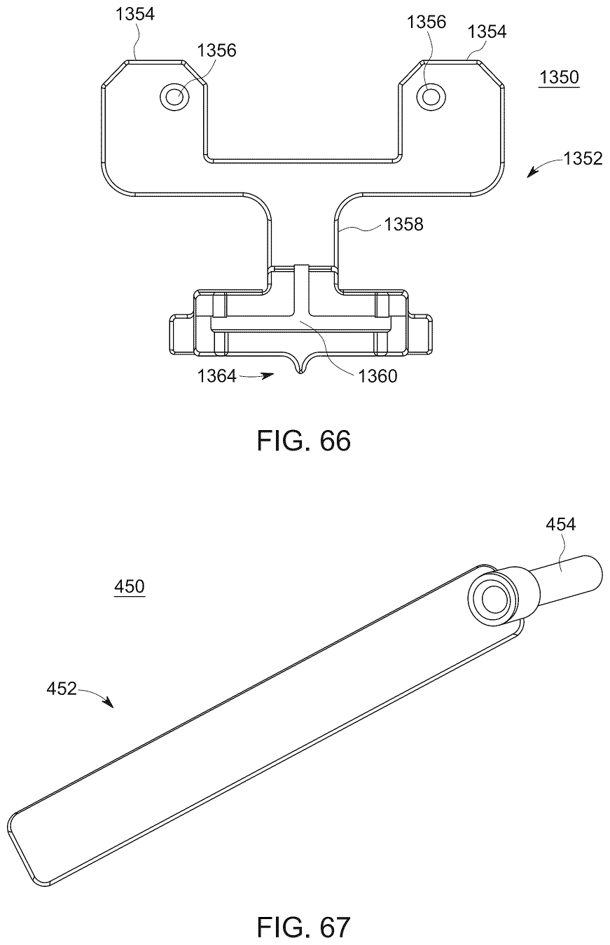

Referring now to FIGS. 59-67, a guide 1300 and components which may be implemented in conjunction with the guide 1300 are shown, according to an exemplary embodiment. The guide 1300 is shown to include a body 1302, with a pair of projections 1304 extending upwardly from the body 1302. As shown, the projections 1304 extend upward from the body 1302 substantially parallel to one another and, as shown, the projections 1304 include substantially the same geometry (although in some aspects the projections 1304 may include alternate geometries from that shown and/or from one another). Each of the projections 1304 are shown to include at least one opening 1306 (e.g., a bore, etc.), where at least one of the openings 1306 is centrally positioned relative to the projection 1304 and extending from a top surface of the guide through to the bottom surface. In some aspects, one or more of the openings 1306 may be biased toward a terminal end of the projections 1304. In some aspects, a second pair of openings 1306 may be disposed within the body 1302 of the guide 1300 and/or partially within the body 1302 of the guide 1300 and partially within the projection 1304. As shown, the openings 1306 disposed within the projections 1304 may have parallel central axes, whereas the second pair of openings 1306 (disposed in the body 1302) may have converging (or diverging) central axes. As shown, each of the openings 1306 include substantially the same size and positioning relative to the projections 1304 and the body 1302. However, in some aspects the openings 1306 may include alternate sizing and/or positioning. As shown, at least two of the openings 1306 are positioned within the projections 1304, however in some aspects one or more openings 1306 may be positioned within the body 1302 adjacent to the base of the projections 1304. In some aspects, one or more pins may be placed within and/or through the openings 1306 and into a bone (e.g., a tibia) so as to couple the guide 1300 with the bone. In some aspects, the guide 1300 may include a coupling element (e.g., a male or female dovetail component, etc.) extending from a portion of the guide 1300 and configured to facilitate releasably coupling of the guide 1300 with other instrumentation and components of a system, for example an ankle arthroplasty system.

The body 1302 is further shown to include an opening 1308 disposed in a central portion thereof. The opening 1308 is shown to have a substantially arced geometry (e.g., curved, radiused, etc.) where an apex of the arced geometry is positioned laterally between the projections 1304. Further, the opening 1308 is shown to include a plurality of scallops disposed within, where the scallops extend from at least one of upper, lower, and lateral sides of the opening 1308, with each of the scallops defining at least a portion (but not necessarily an entirety) of a cylindrical geometry (or other similar geometry) extending from the top surface of the guide 1300 at least partially through to the bottom surface (e.g., through the depth of the opening 1308). Accordingly, as shown in FIGS. 59 and 67, the drill guide 450 of has a complimentary size and geometry to the scallops allowing it may be inserted within each of the scallops so as to guide a drill therein and therethrough such that drilling into the bone with which the guide 1300 is coupled may be accomplished. The opening 1308 includes a substantially elongated, rectangular geometry and is positioned above a central window defined on all sides by components of the guide 1300, with the central window configured to provide visibility of the anatomy (e.g., a joint) beneath the guide 1300 when the guide 1300 is coupled with a bone.

The guide 1300 is further shown to include a pair of legs 1312 extending downward (e.g., in a direction opposite that of the projections) from opposing lateral sides of the body 1302. As shown, the pair of legs 1312 extend at a substantially oblique angle relative to one another. Further, each of the pair of legs 1312 (at a point along a length thereof, for example the terminal end) defines the greatest lateral dimension of the guide 1300 on each side thereof. Each of the pair of legs 1312 are shown to include at least one projection 1314 on an inner surface thereof, positioned substantially below a cross member that is integral with each of the pair of legs 1312 and defining an opening 1310. As shown, at least one of the projections 1314 is substantially triangular in shape and points toward the central opening positioned between the pair of legs 1312 and defined laterally by the legs 1312. Each of the pair of legs is shown to include a slot 1316 disposed therein, wherein the slot 1316 is defined laterally by each of the legs 1312 and vertically in an upward direction by the body 1302, but is undefined (i.e., open) vertically in a lower direction. Accordingly, each of the slots 1316 may be configured to receive a cutting instrument therein (e.g., a reciprocating saw, sagittal saw, etc.) so as to facilitate the cutting/resection of the bone with which the guide 1300 is coupled. As shown, the slots 1316 are positioned substantially parallel to a central axis of each of the pair of legs 1312, however in some aspects the slots 1316 may include alternate geometries.

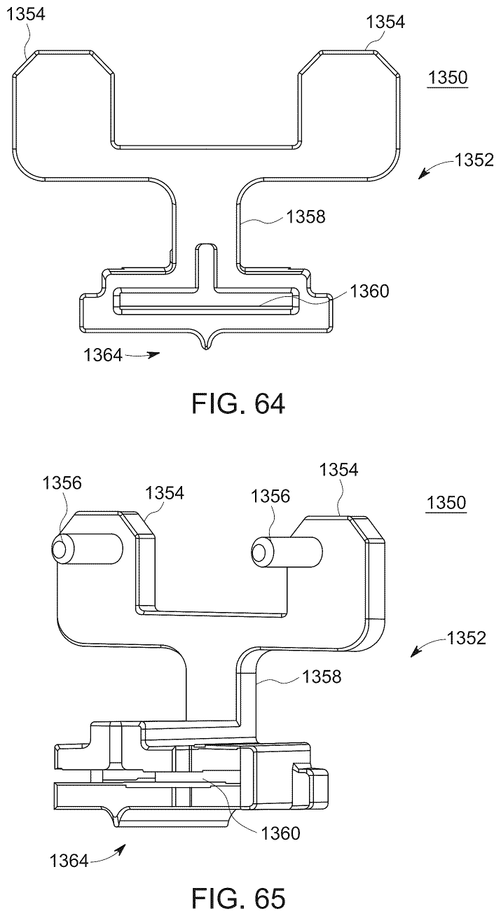

The guide 1300 is shown to be releasably couplable with an instrument 1350 (See FIG. 64) and/or releasably couple with one or more other instruments, for example the instrument 1240 shown in at least FIG. 51. As shown in FIGS. 64-66, the instrument 1350 is shown to include a pair of projections 1354 extending upwardly from a body 1352 substantially parallel to one another. Each of the projections 1354 are shown to include an extension 1356 extending substantially perpendicularly from the bottom surface of the projections 1354, where the extensions 1356 as shown, include a substantially cylindrical geometry with dimensions configured to facilitate reception within at least two of the openings 1306 of the guide 1300. The instrument 1350 is further shown to include a shaft 1358 extending downward from the body 1352 which includes an opening 1360 therein. As shown, the opening 1360 includes both horizontal and vertical rectangular geometries, collectively forming an upside-down “T” shaped opening and configured to receive at least a portion of an instrument therein, for example the instrument 850. Positioned on and extending from a bottom surface of the shaft 1358 (and below the opening 1360) is a protrusion 1364, which extends downward from the shaft 1358.

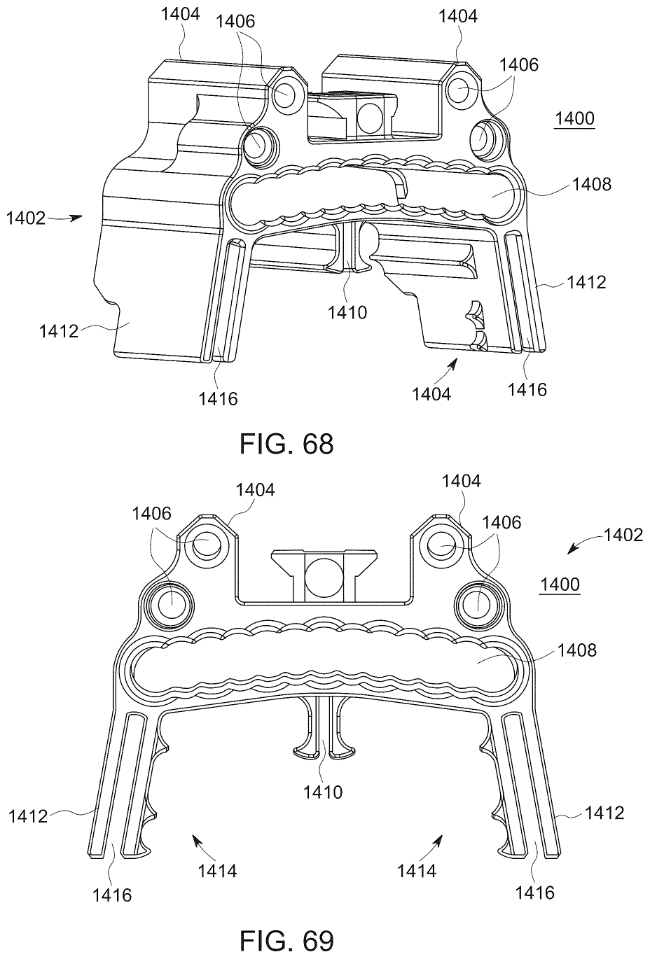

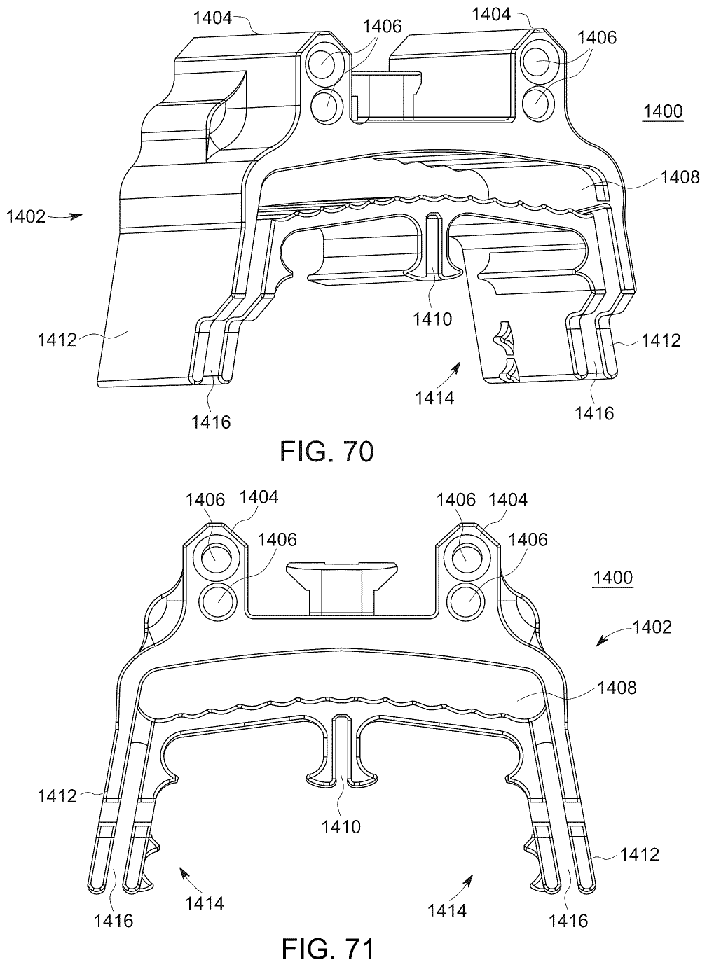

Referring now to FIGS. 68-71, a guide 1400 is shown, according to an exemplary embodiment. The guide 1400 is shown to include a body 1402, with a pair of projections 1404 extending upwardly from the body 1402. As shown, the projections 1404 extend upward from the body 1402 substantially parallel to one another and, as shown, the projections 1404 include substantially the same geometry (although in some aspects the projections 1404 may include alternate geometries from that shown and/or from one another). Each of the projections 1404 are shown to include at least one opening 1406 (e.g., a bore, etc.), where at least one of the openings 1406 is centrally positioned relative to the projection 1404 and extending from a top surface of the guide through to the bottom surface. In some aspects, one or more of the openings 1406 may be biased toward a terminal end of the projections 1404. In some aspects, a second pair of openings 1406 may be disposed within the body 1402 of the guide 1400 and/or partially within the body 1402 of the guide 1400 and partially within the projection 1404. As shown, the openings 1406 disposed within the projections 1404 may have parallel central axes, whereas the second pair of openings 1406 (disposed in the body 1402) may have converging (or diverging) central axes. As shown, each of the openings 1406 include substantially the same size and positioning relative to the projections 1404 and the body 1402. However, in some aspects the openings 1406 may include alternate sizing and/or positioning. As shown, at least two of the openings 1406 are positioned within the projections 1404, however in some aspects one or more openings 1406 may be positioned within the body 1402 adjacent to the base of the projections 1404. In some aspects, one or more pins may be placed within and/or through the openings 1406 and into a bone (e.g., a tibia) so as to couple the guide 1400 with the bone. In some aspects, the guide 1400 may include a coupling element (e.g., a male or female dovetail component, etc.) extending from a portion of the guide 1400 and configured to facilitate releasably coupling of the guide 1400 with other instrumentation and components of a system, for example an ankle arthroplasty system.

The body 1402 is further shown to include an opening 1408 disposed in a central portion thereof. The opening 1408 is shown to have a substantially arced geometry (e.g., curved, radiused, etc.) where an apex of the arced geometry is positioned laterally between the projections 1404. Further, the opening 1408 is shown to include a plurality of scallops disposed within, where the scallops extend from at least one of upper, lower, and lateral sides of the opening 1408, with each of the scallops defining at least a portion (but not necessarily an entirety) of a cylindrical geometry (or other similar geometry) extending from the top surface of the guide 1400 at least partially through to the bottom surface (e.g., through the depth of the opening 1408). Accordingly, the drill guide (See FIG. 67) has a complimentary size and geometry to the scallops and may be inserted within each of the scallops so as to guide a drill therein and therethrough such that drilling into the bone with which the guide 1400 is coupled may be accomplished. The opening 1408 includes a substantially elongated, rectangular geometry and is positioned above a central window defined on all sides by components of the guide 1400, with the central window configured to provide visibility of the anatomy (e.g., a joint) beneath the guide 1400 when the guide 1400 is coupled with the bone.

The guide 1400 is further shown to include a pair of legs 1412 extending downward (e.g., in a direction opposite that of the projections) from opposing lateral sides of the body 1402. As shown, the pair of legs 1412 extend at a substantially oblique angle relative to one another. Further, each of the pair of legs 1412 (at a point along a length thereof, for example the terminal end) defines the greatest lateral dimension of the guide 1400 on each side thereof. Each of the pair of legs 1412 are shown to include at least one projection 1414 on an inner surface thereof. As shown, the at least one of the projections 1414 is substantially triangular shape and points toward the central opening positioned between the pair of legs 1412 and defined laterally by the legs 1412. Further, the guide 1400 is shown to include a pair of protrusions extending downward from the body 1402 and at least partially defining an opening 1410 there between. Each of the pair of legs is shown to include a slot 1416 disposed therein, wherein the slot 1416 is defined laterally by each of the legs 1412 and vertically in an upward direction by the body 1402, but is undefined (i.e., open) vertically in a lower direction. Accordingly, each of the slots 1416 may be configured to receive a cutting instrument therein (e.g., a reciprocating saw, sagittal saw, etc.) so as to facilitate the cutting/resection of the bone with which the guide 1400 is coupled. As shown, the slots 1416 are positioned substantially parallel to a central axis of each of the pair of legs 1412, however in some aspects the slots 1416 may include alternate geometries.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed:

1. An orthopedic instrument system, comprising:
a resection guide releasably couplable with a bone of a patient, the resection guide comprising:
a pair of openings disposed in the resection guide and extending from a top surface through to a bottom surface of the resection guide;
an elongated opening disposed in the body and beneath the pair of openings, wherein the elongated opening comprises at least one scalloped opening therein; and
a pair of legs extending substantially downward from the body of the resection guide, wherein each of the legs comprises a slot, wherein the slot opens in at least one direction; and
a drill guide configured to be inserted into and removed from each of the at least one scalloped openings.

2. The orthopedic instrument system of claim 1, wherein the pair of legs are positioned at an angle relative to one another.

3. The orthopedic instrument system of claim 1, wherein each of the openings comprises a central axis, wherein the central axes of the openings are substantially parallel.

4. The orthopedic instrument system of claim 3, further comprising a second pair of openings disposed within the body of the resection guide and extending from the top surface through to the bottom surface thereof.

5. The orthopedic instrument system of claim 4, wherein each of the second pair of openings comprises a central axis, wherein the central axes of the second pair of openings are either converging or diverging.

6. The orthopedic instrument system of claim 1, wherein the elongated opening comprises a substantially arced geometry.

7. The orthopedic instrument system of claim 6, further comprising a pair of projections extending substantially upward from the body of the resection guide.

8. The orthopedic instrument system of claim 7, wherein each of the openings are disposed within each of the pair of projections.

9. The orthopedic instrument system of claim 8, wherein the apex of the elongated opening is positioned within the body and laterally between each of the pair of projections.

10. A resection guide releasably couplable with a bone of a patient, the resection guide comprising:
a pair of projections extending upward from a body of the resection guide, each of the pair of projections comprising an opening disposed therein and extending from a top surface through to a bottom surface of the resection guide;
a first elongated opening disposed in the body and beneath the pair of openings, wherein the elongated opening comprises at least one scalloped opening therein; and
a pair of legs extending substantially downward from the body of the resection guide, wherein each of the pair of legs comprises a slot, wherein the slot is open in at least one direction.

11. The resection guide of claim 10, further comprising a second pair of openings disposed within the body of the resection guide.

12. The resection guide of claim 11, wherein each opening of the second pair of openings comprises a central axis, wherein the central axes are one of converging or diverging.

13. The resection guide of claim 10, wherein each of the openings comprise a central axis, wherein the central axes are substantially parallel to one another.

14. The resection guide of claim 10, wherein the at least one scalloped opening defines at least a portion of a substantially cylindrical geometry extending from the top surface of the resection guide and at least partially through to the bottom surface of the resection guide.

15. The resection guide of claim 10, wherein the pair of legs extend from the body at a substantially oblique angle.

16. The resection guide of claim 10, wherein the first elongated opening has a substantially curved geometry.

17. The resection guide of claim 16, wherein an apex of the first elongated opening is positioned laterally between the pair of projections.

18. The resection guide of claim 10, further comprising a second elongated opening positioned below the first elongated opening.

19. The resection guide of claim 18, wherein the second elongated opening is defined laterally by each of the pair of legs.

20. The resection guide of claim 19, wherein the second elongated opening is configured to receive at least a portion of a cutting tool therein.
